(19) United States
(12) Patent Application Publication
(10) Pub. No.: US 2021/0298819 A1
(43) Pub. Date: Sep. 30, 2021

(54) SURGICAL INTRODUCER WITH TISSUE TREATMENT WINDOW

(71) Applicant: Daniel Ezra Walzman, Bergenfield, NJ (US)

(72) Inventor: Daniel Ezra Walzman, Bergenfield, NJ (US)

(21) Appl. No.: 17/308,133

(22) Filed: May 5, 2021

Related U.S. Application Data
(63) Continuation-in-part of application No. 16/602,133, filed on Aug. 12, 2019, now Pat. No. 11,065,021.
Application No. 16/602,133 is a continuation-in-part of application No. 16/151,335, filed on Oct. 3, 2018, now abandoned.

(51) Int. Cl. A61B 18/14

(52) U.S. Cl. CPC A61B 18/14; A61B 2018/00327

(57) ABSTRACT

A system is disclosed for performing a surgical procedure on a patient. The osteotomy system includes an elongated introducer defining a lumen and including a window formed in an outer wall thereof that is in communication with the lumen. At least one medical instrument is configured for insertion into the patient through the elongated introducer such that the at least one medical instrument is positionable within the window. The window is configured to receive target tissue such that the target tissue extends into the lumen, whereby the elongated introducer shields collateral tissue from the at least one medical instrument to reduce any unintended effect on the collateral tissue during the surgical procedure.

### CROSS-REFERENCE TO RELATED APPLICATION(S)

This application is a continuation-in-part of application Ser. No. 16/602,133, filed Aug. 12, 2019, which is a continuation-in-part of application Ser. No. 16/151,335, filed Oct. 3, 2018. The entire contents of each of these applications are incorporated herein by reference.

### 1. Field of the Invention

The present invention relates generally to medical devices, systems, and methods, and more particularly, to a surgical introducer with a window for surgical instrumentation to treat various disorders such as disorders of the paranasal sinuses, ears, nose, and/or throat (ENT).

### 2. Background of the Related Art

Image-guided surgical techniques and devices were developed for neurosurgery and have now been adapted for use in certain ear, nose and throat surgeries, including sinus surgeries. See, Kingdom T. T., Orlandi R. R., Image-Guided Surgery of the Sinuses: Current Technology and Applications, Otolaryngol. Clin. North Am. 37(2):381-400 (April 2004). Generally, image-guided surgery involves obtaining images prior to surgery and then using the images to help the surgeon perform the surgery. Often, during the course of image guided surgery, electromagnetic sensors/tracking systems and/or radiofrequency electromagnetic sensors (e.g., electromagnetic coils) are placed on the surgical instruments and on a localizer frame worn by the patient.

U.S. Patent Application Publication No. 20090216196 to Drontle et al. discloses an apparatus and method for accessing a sinus cavity. Drontle does not disclose a unitary, single lumen or a window (e.g., a side-slit) feature and lacks a built-in imaging system (e.g., one or more cameras).

U.S. Pat. No. 6,527,704 to Chang et al. discloses an endoscopic camera system integrated with a trocar sleeve, having a side groove that channels devices parallel to the catheter. The present disclosure describes a different structure, a window (e.g. a side-slit) which channels devices perpendicular to the body of the device.

U.S. Patent Application Publication No. 2015/0141755 to Tesar discloses surgical visualization systems. Tesar discloses a plurality of cameras, but does not disclose the placement of at least two cameras in specific positions.

Osteotomy is a surgical procedure that is performed to treat (e.g., cut, reshape, excise, or otherwise alter) tissue (e.g. bone), to repair a damaged joint, or to shorten or lengthen a bone (e.g., to change alignment of the bone). Osteotomy systems typically include several standalone devices that perform different functions including, for example, cauterization, irrigation, aspiration, cutting, and imaging, among others, which are generally used in pairs and occasionally in threes. Imaging during an osteotomy procedure conventionally includes the use of external sensors with tagged instruments and/or image-collection, either prior to or during the surgical procedure. For example, a separate instrument supporting one or more imaging devices is often inserted into the patient to allow images of the target area to be captured during the course of the osteotomy procedure.

The lack of osteotomy devices that provide built-in irrigation and/or aspiration systems requires the insertion and removal of separate systems, which not only delays the surgical procedure, but results in movement of the sensor(s) mounted on proximal portions of the instruments (e.g., on the handpiece) to be moved away from the target area and, thus, a reduction in the accuracy of the information provided by the sensor(s). The use of multiple, separate systems also increases the overall amount of anatomical space required, resulting in a more invasive patient experience that often requires the administration of general anesthesia, particular in the context of sinus surgery and other ENT procedures.

The present disclosure addresses this shortcoming, among others, and describes minimally invasive systems, devices, and surgical procedures that may not only eliminate the need for general anesthesia (and the associated risks), but reduce the complexity, time, and overall cost of the surgical procedures, as well as any iatrogenic impact and the patient's recovery time. By reducing invasiveness and obviating the need for general anesthesia, it is envisioned that the surgical procedures described herein may be performed in an office setting, rather than in a hospital operating room or other such surgical theater. While select balloon sinuplasty procedures are sometimes performed in such (nonhospital) settings, these procedures are less effective at relieving symptoms and carry an elevated risk of recurrent symptomatic sinus blockages and/or infections.

The systems, devices, and surgical procedures described herein also protect (shield) the collateral tissue adjacent to (surrounding) the tissue that is the target of the surgical procedure to reduce (if not entirely eliminate) any iatrogenic impact on the collateral tissue that may otherwise result during treatment (e.g., cutting, reshaping, excision, or other such alteration of the target tissue). While systems, devices, and surgical procedures are known that offer some protection of collateral tissue, the techniques used to shield collateral tissue are often cumbersome and employ devices which are not only difficult to position, but tend to migrate out of position, which can cause tissue irritation and other complications.

### SUMMARY OF THE INVENTION

The present disclosure describes various systems, devices, and methods that find wide applicability in a variety of minimally invasive surgical procedures, particularly in the context of osteotomies, sinus procedures, and other ENT procedures. The systems and devices described herein include an elongated member (e.g., an introducer, a sheath, or the like) that facilitates the introduction of various medical instruments therethrough (e.g., a tissue cutter, an imaging device, a cauterization device, an aspiration device, and/or an irrigation device) to a surgical site to treat (e.g., cut, reshape, excise, or otherwise alter) target tissue. The systems and devices described herein facilitate the simultaneous treatment, irrigation, and aspiration of the target tissue while providing live, visual information to the clinician by integrating multifunctionality into a single system (device). The target tissue can include for example bone, soft tissue, calcified tissue, mucosa, etc.

In one aspect of the present disclosure, an osteotomy device is described that includes an integrated imaging system (e.g., one or more imaging devices that are supported on or are movable through the osteotomy device) and a window defining a recessed treatment area (chamber) that is configured to receive (accommodate) the target tissue to thereby shield and protect the collateral tissue and reduce (if not entirely prevent) any iatrogenic impact on the collateral tissue that may otherwise result during treatment of the target tissue. To increase control and/or accuracy during the surgical procedure, it is also envisioned that the systems and devices described herein may be configured for deflection (reconfiguration) to improve navigation through the patient's anatomy (e.g., the patient's sinuses). For example, in various embodiments, it is envisioned that the systems and devices described herein may be malleable (e.g., bendable) and/or steerable.

In another aspect of the present disclosure, an osteotomy system is disclosed for performing a surgical procedure on a patient. The osteotomy system includes an elongated introducer defining a lumen and including a window formed in an outer wall thereof that is in communication with the lumen, a first imaging device that is fixedly supported on the elongated introducer distally of the window, a second imaging device that is fixedly supported on the elongated introducer proximally of the window, and at least one medical instrument that is configured for insertion into the patient through the elongated introducer such that the at least one medical instrument is positionable within the window. The window is configured to receive target tissue such that the target tissue extends into the lumen, whereby the elongated introducer shields collateral tissue from the at least one medical instrument to reduce any unintended effect on the collateral tissue during the surgical procedure.

In certain embodiments, the first imaging device and the second imaging device may be embedded in the outer wall of the elongated introducer.

In certain embodiments, the at least one medical instrument may include a tissue treatment device (e.g., a tissue cutter, a drill, etc.) that is configured for insertion through the lumen of the elongated introducer and into the window to facilitate treatment of the target tissue when the target tissue is positioned within the window.

In certain embodiments, the at least one medical instrument may further include an aspiration system that extends into the patient through the elongated introducer such that the aspiration system is integrated into the elongated introducer.

In certain embodiments, the at least one medical instrument may further include an irrigation system that extends into the patient through the elongated introducer such that the irrigation system is integrated into the elongated introducer.

In certain embodiments, the at least one medical instrument may further include a cauterization system that extends into the patient through the elongated introducer such that the cauterization system is integrated into the elongated introducer.

In certain embodiments, the osteotomy system may further include a handle that is connected to the elongated introducer.

In certain embodiments, the handle may be fixedly connected to the elongated introducer.

In certain embodiments, the handle may be configured for disconnection from and reconnection to the elongated introducer.

In certain embodiments, the handle may include a first port that is configured to receive the aspiration system such that the aspiration system extends into the elongate introducer through the first port, a second port that is configured to receive the irrigation system such that the irrigation system extends into the elongate introducer through the second port, and a third port that is configured to receive the cauterization system such that the cauterization system extends into the elongate introducer through the third port.

In certain embodiments, the elongated introducer may define a transverse cross-sectional dimension (e.g., a diameter) that is (generally) uniform between the proximal and distal ends of the elongated introducer.

In certain embodiments, a second medical instrument is configured for insertion into the patient through the elongated introducer such that the second medical instrument is extendable distal of a distal end hole of the introducer and visualized by the first imaging device.

In another aspect of the present disclosure, an osteotomy system is disclosed for use in performing a surgical procedure on a patient. The osteotomy system includes an elongated introducer, a first imaging device, a second imaging device, and at least one medical instrument. The elongated introducer includes a window that is configured to receive target tissue such that the target tissue extends into the elongated introducer and a visualization port that is located proximally of the window. The first imaging device is positioned adjacent to the visualization port such that the target tissue is viewable through the visualization port via the first imaging device and the second imaging device is positioned distally of the window. The at least one medical instrument is configured for insertion into the patient through the elongated introducer such that the at least one medical instrument is positionable within the window. The window is configured to receive the target tissue such that the target tissue extends into the elongated introducer, whereby the elongated introducer shields collateral tissue adjacent to the target tissue from the at least one medical instrument to reduce any unintended effect on the collateral tissue during the surgical procedure.

In certain embodiments, the first imaging device may extend through the visualization port such that the first imaging device protrudes laterally from the elongated introducer.

In certain embodiments, the first imaging device and the second imaging device may be embedded in an outer wall of the elongated introducer.

In certain embodiments, the elongated introducer may include a primary lumen, a first ancillary lumen that is discrete from the primary lumen, a second ancillary lumen that is discrete from the primary lumen and the first ancillary lumen, and a third ancillary lumen that is discrete from the primary lumen, the first ancillary lumen, and the second ancillary lumen.

In certain embodiments, the osteotomy system may further include a tissue treatment device (e.g., a tissue cutter, a drill, etc.) that is configured for insertion into the primary lumen such that the tissue treatment device is positionable within the window to treat the target tissue, an irrigation system that extends through the first ancillary lumen such that the irrigation system is integrated into the elongated introducer, e.g., built into the wall, an aspiration system that extends through the second ancillary lumen such that the aspiration system is integrated into the elongated introducer, and a cauterization system that extends through the third ancillary lumen such that the cauterization system is integrated into the elongated introducer.

In certain embodiments, the osteotomy system may further include a handle that is connected to the elongated introducer (either fixedly or releasably such that the handle is disconnectable from and reconnectable to the elongated introducer).

In certain embodiments, the handle may include a first port that is configured to receive the irrigation system such that the irrigation system extends into the elongated introducer through the first port, a second port that is configured to receive the aspiration system such that the aspiration system extends into the elongated introducer through the second port, and a third port that is configured to receive the cauterization system such that the cauterization system extends into the elongated introducer through the third port.

In another aspect of the present disclosure, a method of performing an osteotomy procedure on a patient's sinuses is disclosed that includes inserting an elongated introducer defining a window that is configured to receive target tissue into the patient's sinuses, visualizing tissue using an imaging device that extends through the elongated introducer and is positioned proximally of the window, inserting a tissue treatment device into the patient's sinuses through the elongated introducer such that the tissue treatment device is positioned within the window, positioning the elongated introducer such that the target tissue is received within the window and extends into the elongated introducer to thereby shield collateral tissue from the tissue treatment device to reduce any unintended effect on the collateral tissue during the osteotomy procedure, and altering the target tissue using the tissue treatment device.

In some embodiments, the method allows safe minimally invasive permanent expansion of a paranasal sinus (such as the maxillary sinus) by removing bone and mucosa around the ostium to enlarge it.

In certain embodiments, the method may further include inserting an irrigation system into the patient's sinuses through the elongated introducer such that the irrigation system extends into the window.

In certain embodiments, the method may further include inserting an aspiration system into the patient's sinuses through the elongated introducer such that the aspiration system extends into the window.

In certain embodiments, the method may further include simultaneously applying irrigation and aspiration during alteration of the target tissue via the tissue treatment device.

### BRIEF DESCRIPTION OF THE DRAWINGS

The present disclosure will be better understood, and objects other than those set forth above will become apparent when, consideration is given to the detailed description and the drawings.

FIG. 1 is a side view of a surgical (osteotomy) system including an osteotomy device according to one embodiment of the present disclosure.

FIG. 2 is a partial, side view of the osteotomy device.

FIG. 3 is a partial, side view of the osteotomy device according to an alternate embodiment of the present disclosure including a door shown in a (partially) open position.

FIG. 4 is partial, side view of the osteotomy device seen in FIG. 3 with the door shown in a closed position.

FIG. 5 is a side view of a tissue cutter for use with the osteotomy device.

FIG. 6A is a partial, side view of the osteotomy device illustrating the treatment of target tissue.

FIG. 6B is a partial, side view of the osteotomy device seen in FIG. 6A.

FIG. 7 is a side view of the osteotomy device according to an alternate embodiment of the present disclosure.

FIG. 8 is a partial, bottom view of the osteotomy device according to an alternate embodiment of the present disclosure.

FIG. 9 is a side view of the osteotomy device according to an alternate embodiment of the present disclosure.

FIG. 10 is a side view of the osteotomy device according to an alternate embodiment of the present disclosure.

FIG. 11 is a side view of the osteotomy device according to an alternate embodiment of the present disclosure.

FIG. 12 is a side view of the osteotomy device according to an alternate embodiment of the present disclosure.

FIG. 13A is a side view of the osteotomy device according to an alternate embodiment of the present disclosure.

FIG. 13B is a partial, bottom view of the osteotomy device seen in FIG. 13A.

FIG. 14A is a side view of the osteotomy device according to an alternate embodiment of the present disclosure.

FIG. 14B is a partial, side view of the osteotomy device seen in FIG. 14A.

FIG. 15 is a side view illustrating use of the surgical system during a surgical (sinus) procedure.

FIG. 16 is a side view of the osteotomy device according to an alternate embodiment of the present disclosure and shown in a first configuration.

FIG. 17 is a transverse, cross-sectional view of the osteotomy device taken along line 17-17 in FIG. 16.

FIG. 18 is a side view of the osteotomy device seen in FIG. 16 shown in a second configuration.

FIG. 19A is a partial, side view of the osteotomy device according to an alternate embodiment of the present disclosure.

FIG. 19B is a partial, bottom view of the osteotomy device seen in FIG. 19A.

FIG. 19C is a transverse, cross-sectional view of the osteotomy device taken along line 19C-19C in FIG. 19A.

FIG. 20 is a partial, side view of the osteotomy device according to an alternate embodiment of the present disclosure.

FIG. 20B is a partial, side view of the osteotomy device seen in FIG. 20A.

FIG. 20C is a transverse, cross-sectional view of the osteotomy device taken along line 20C-20C in FIG. 20A.

FIG. 21A is a partial, side view of the osteotomy device according to an alternate embodiment of the present disclosure.

FIG. 21B is a partial, side view of the osteotomy device seen in FIG. 21A.

FIG. 21C is a transverse, cross-sectional view of the osteotomy device taken along line 21C-21C in FIG. 21A.

FIG. 21D is a transverse, cross-sectional view of an alternate embodiment of the osteotomy device seen in FIG. 21A.

FIG. 22A is a partial, side view of the osteotomy device according to an alternate embodiment of the present disclosure.

FIG. 22B is a transverse, cross-sectional view of the osteotomy device taken along line 22B-22B in FIG. 22A.

FIG. 22C is a transverse, cross-sectional view of an alternate embodiment of the osteotomy device seen in FIG. 22A.

FIG. 23A is a partial, side view of the osteotomy device according to an alternate embodiment of the present disclosure.

FIG. 23B is a transverse, cross-sectional view of the osteotomy device taken along line 23B-23B in FIG. 23A.

FIG. 23C is a partial, side view of the osteotomy device seen in FIG. 23A according to an alternate embodiment of the present disclosure.

### DETAILED DESCRIPTION OF THE INVENTION

Referring now to the drawings, wherein like reference characters identify similar structural components and features, various embodiments of the present disclosure will be described. As used herein, the term “proximal” should be understood as referring to that portion or section of the pertinent structure that is closer to the user during proper use and the term “distal” should be understood as referring to that portion or section of the pertinent structure that is further from to the user during proper use. Additionally, the terms “apparatus,” “instrument,” “device,” and “system” (and variations thereof), and the terms “hole,” “aperture,” and “opening” (and variations thereof) may be used interchangeably herein.

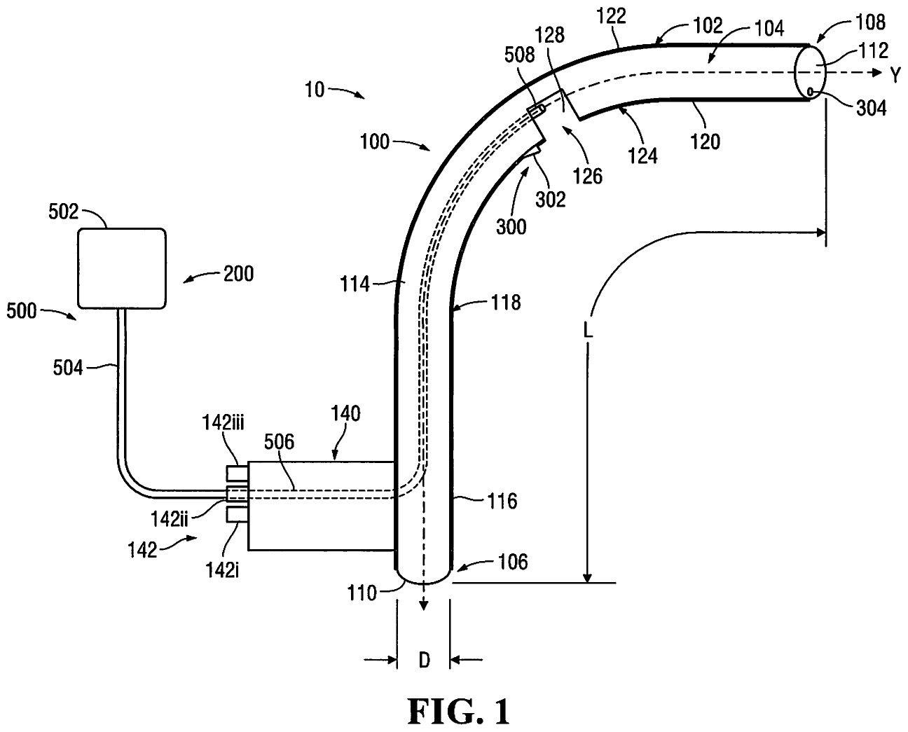

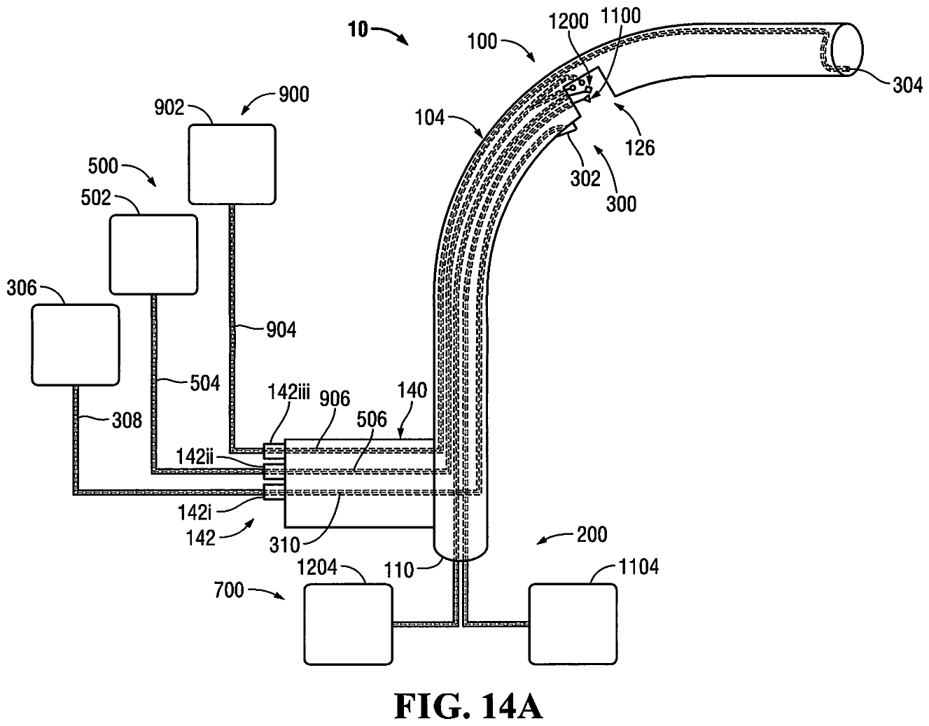

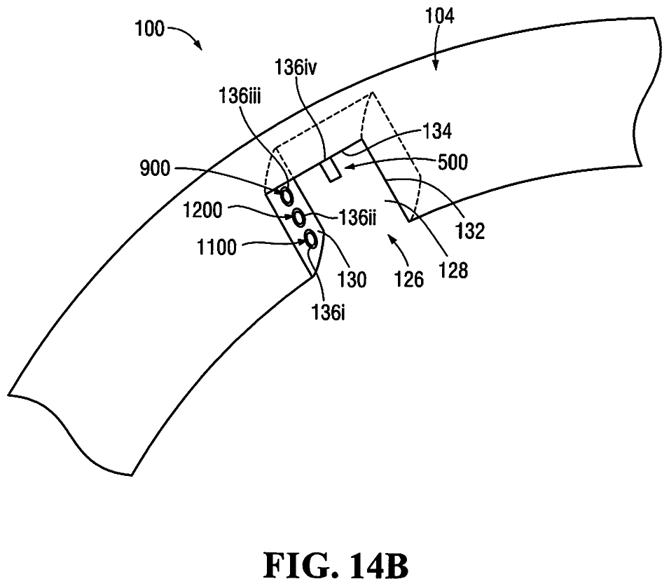

With reference to FIGS. 1-15, a surgical (osteotomy) system 10 is disclosed that includes an osteotomy device 100 having an elongated introducer 102 (also referred to herein as a sheath, a member, an element, or a protector) with a tubular body 104 that is (generally) cylindrical in configuration. The tubular body 104 includes respective proximal and distal ends 106, 108 and defines a longitudinal axis Y, an overall axial (longitudinal) length L that extends along the longitudinal axis Y between the ends 106, 108, and a transverse cross-sectional dimension (e.g., a diameter) D that is (generally) uniform (e.g., constant, non-variable) along the length L of the tubular body 104 (e.g., between the ends 106, 108). Depending upon the particular context of the surgical procedure in which the osteotomy device 100 is employed, the patient's anatomy, etc., embodiments are also envisioned in which the transverse cross-sectional dimension D may vary along the length L of the tubular body 104.

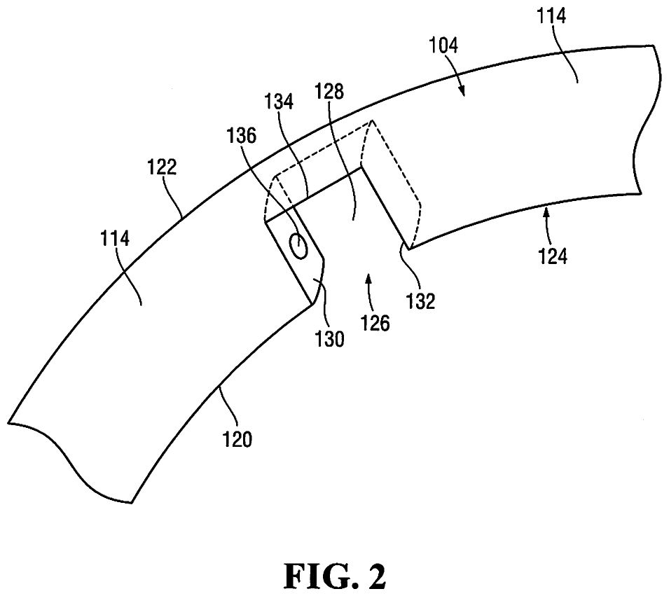

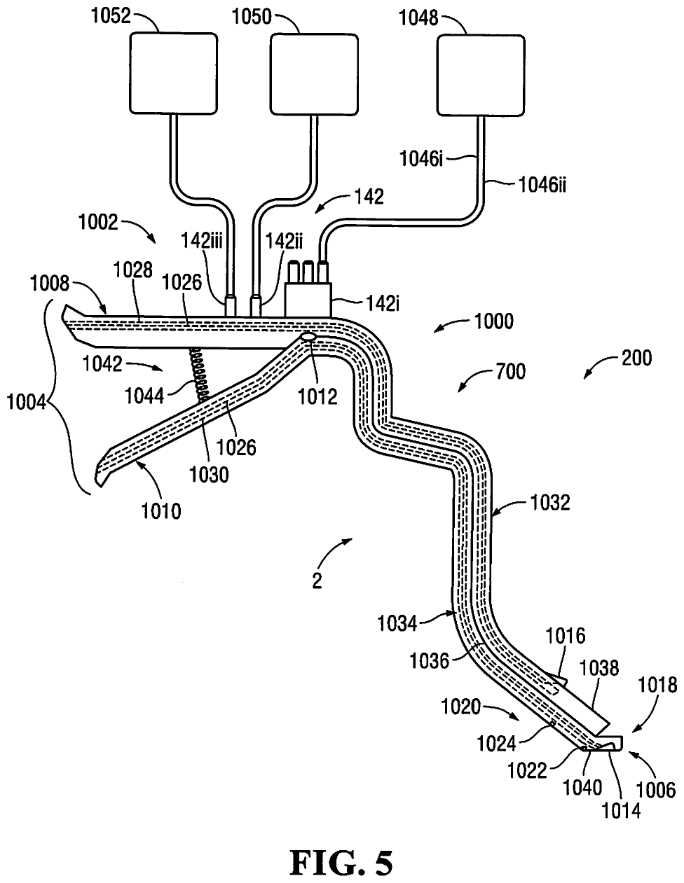

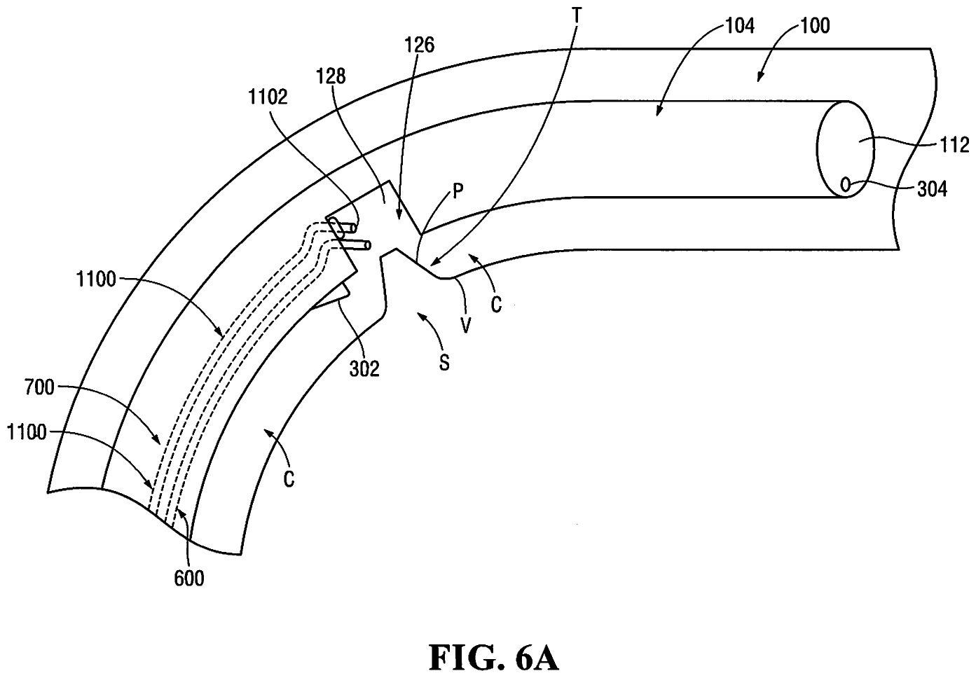

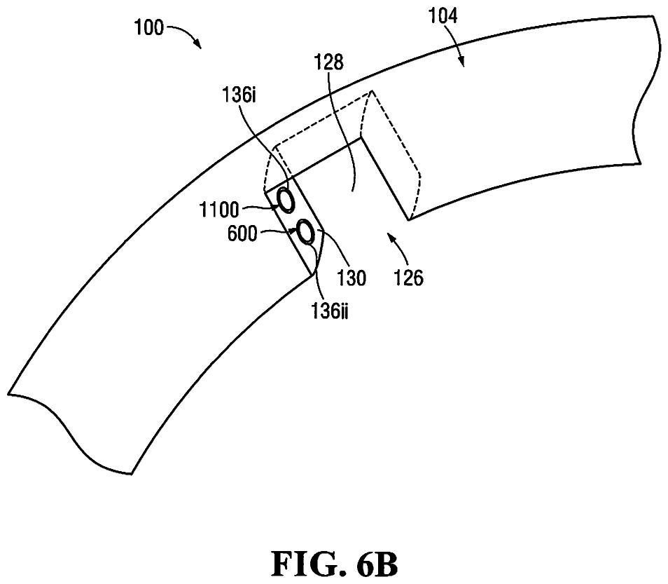

The tubular body 104 includes a proximal opening (end hole) 110 (e.g., located outside the patient's body), a distal opening (end hole) 112 (e.g., located within the patient's body), and one or more lumens 114 (FIG. 2) (protection conduits, channels) that extend through the tubular body 104 between the openings 110, 112 (for simplicity and clarity, the lumen(s) 114 are not shown in FIG. 1). The lumen(s) 114 are configured to receive one or more medical instruments 200 (FIG. 5) such that the medical instrument(s) 200 are insertable through the osteotomy device 100 and into the patient to treat (e.g., cut, reshape, excise, or otherwise alter) target tissue T (FIG. 6A) (e.g., bone, soft tissue, calcified tissue, mucosa, etc.) at a surgical site S that is the focus of the surgical procedure. It is envisioned that the medical instrument(s) 200 may include a tissue treatment system or device, an imaging system or device, an aspiration system or device, a cauterization system or device, and/or an irrigation system or device, examples of which are described herein below. It is also envisioned that the medical instrument(s) 200 may include a Kerrison bone punch or rongeur (either manually operated or powered) or any other medical instrument or device required during the surgical procedure, either individually or in combination.

In the particular embodiment seen in FIG. 1, for example, the tubular body 104 includes a single primary (working) lumen 114 that is configured to receive each of the medical instruments 200 used during the course of the surgical procedure. As elaborated upon below, however, in various embodiments of the disclosure, it is envisioned that the tubular body 104 may include one or more ancillary lumens that extend in discrete, e.g., (generally) parallel relation to the primary lumen 114, which allows for the insertion of different medical instruments 200 through the primary lumen and/or one or more of the ancillary lumens.

Advancing the medical instrument(s) 200 to the surgical site S through the tubular body 104, as opposed to externally of the osteotomy device 100, allows for the elimination of external systems or devices. The elimination of external systems and devices lessens the space required by the surgical system 10 and allows the surgical procedure to be conducted in a minimally invasive manner that reduces any iatrogenic impact on the patient and, thus, the patient's recovery time, while simplifying the surgical procedure by confining the medical instrument(s) to a smaller, more efficient operative space within the tubular body 104. Advancing the medical instrument(s) 200 through the tubular body 104 also protects the medical instrument(s) 200 from the patient's anatomy, blockages, debris, etc., that may otherwise damage the medical instrument(s) 200 (e.g., imaging devices or other such sensitive or delicate components) and guides the medical instrument(s) 200 so as to achieve and/or maintain the medical instrument(s) 200 in a particular orientation.

The tubular body 104 may include any suitable biocompatible material or combination of materials. For example, it is envisioned that the tubular body 104 may include (e.g., may be formed partially or entirely from) one or more metallic materials, plastic materials, polymeric materials, composite materials, etc. It is envisioned that the tubular body 104 may be flexible and/or resilient in construction to facilitate the navigation of various cavities and/or channels in the patient's anatomy (e.g., the patient's sinuses) and access to the target tissue T. To assist insertion and advancement of the tubular body 104 through the patient's body and further inhibit (if not entirely prevent) irritation and/or damage to the patient and/or the medical instrument(s) 200, it is envisioned that the tubular body 104 may include a protective outer coating 116 (e.g., a hydrogel or the like) on an outer surface 118 thereof. In such embodiments, it is envisioned that the coating 116 may extend along a portion of the axial length L of the tubular body 104 or along the entirety of the axial length L.

In the particular embodiment illustrated, the tubular body 104 includes a non-linear configuration having a (pre-formed) curvature that defines an inner curved surface 120 and an outer curved surface 122. Embodiments of the tubular body 104 including a (generally) linear configuration devoid of any pre-formed curvature, however, are also envisioned herein.

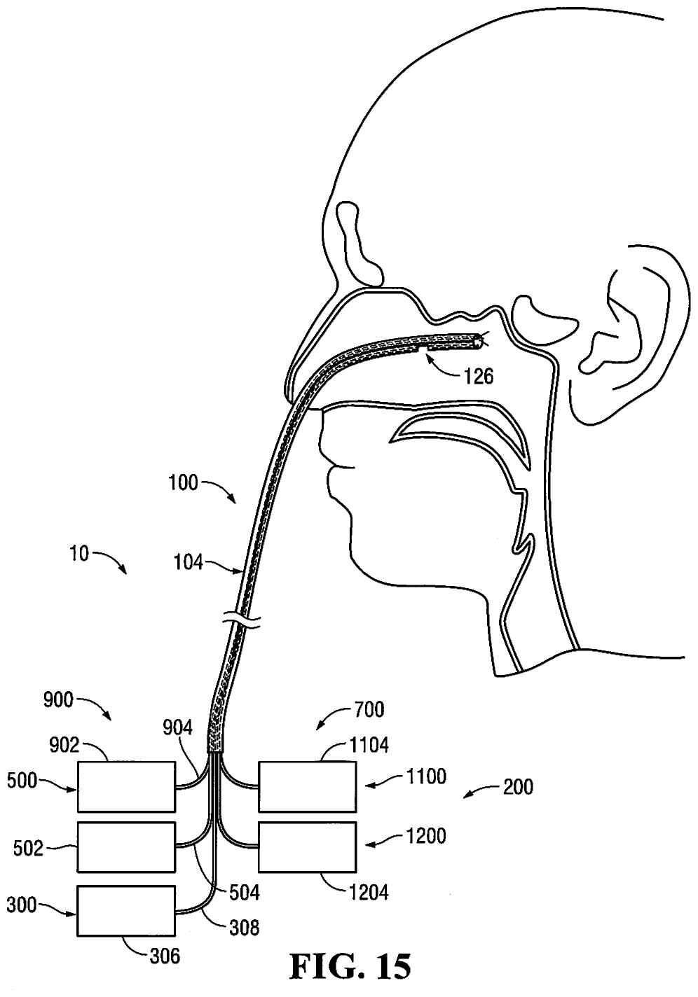

The tubular body 104 includes an outer wall 124 with a window 126 (FIGS. 1, 2) formed therein (e.g., on the inner curved surface 120) between the proximal opening 110 and the distal opening 112. The window 126 defines a treatment area 128 (also referred to herein as a protection area, a shielding area, or a protection chamber) that is recessed into the tubular body 104 (e.g., such that the window 126 communicates with the primary lumen 114). The window 126 (e.g., the treatment area 128) is configured to (at least partially) receive the target tissue T (FIG. 6A) during the surgical procedure and may include any structure suitable for that intended purpose. By positioning the target tissue T within the window 126 and focusing treatment to the treatment area 128, the tubular body 104 functions to shield (protect) collateral tissue C surrounding (e.g., adjacent to) the target tissue T, which reduces (if not entirely eliminates) any unintended iatrogenic impact on the collateral tissue C that may otherwise occur during treatment of the target tissue T. The window 126 in the illustrated embodiment is spaced from the distal opening 112 along the curve of the tubular body 104 so the window 126 and distal opening 112 are in a non-linear arrangement, i.e., a length between the window 126 and distal opening 112 is defined along a non-linear path. In other embodiments, the curvature of the tubular body 104 and/or the spacing between the window and distal hole can be different so that they are in a linear or more linear arrangement. FIG. 15 discussed below shows the latter arrangement for use in a sinus procedure, however, the former arrangement (non-linear) is also contemplated. The proximal and distal imaging devices discussed herein, can likewise be spaced about the curvature of the tubular body 104 defined by a non-linear arrangement, i.e., a length between the two imaging devices is defined along a non-linear path or alternatively, due to the curvature of the tube 104 and/or the spacing, be in a linear or more linear arrangement.

Depending upon the particular context of the surgical procedure, the patient's anatomy, the configuration of the target tissue T, the location of the target tissue T, etc., it is envisioned that the window 126 (and the treatment area 128) may include any suitable configuration. For example, it is envisioned that the window 126 be configured as a side hole, an opening, an aperture, a side slit, a cutout, or the like. Additionally, although envisioned as including a (generally) annular (e.g., circular) cross-sectional configuration, it should be appreciated that the particular geometrical configuration of the window 126 may be varied without departing from the scope of the present disclosure. For example, it is envisioned that the window 126 may including a cross-sectional configuration that is (generally) rectangular, (generally) square, (generally) elliptical, (generally) linear, etc.

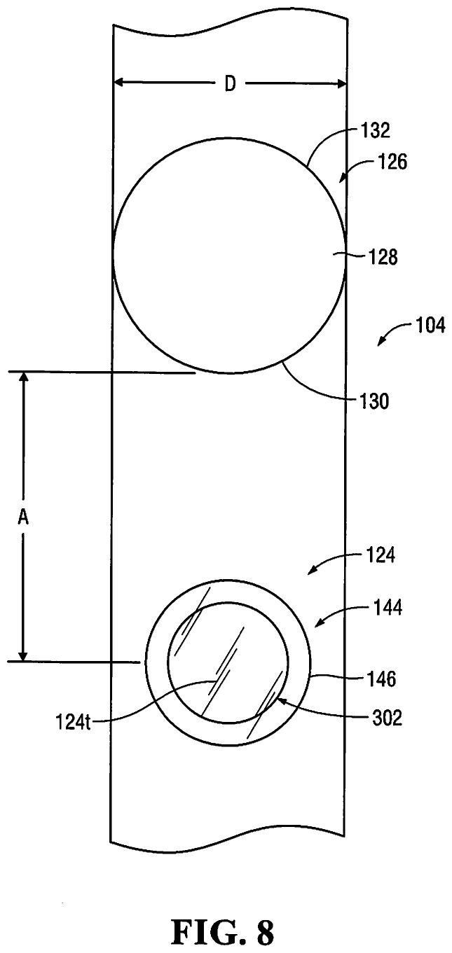

As seen in FIG. 8, for example, the window 126 may span the entire transverse cross-sectional dimension D of the tubular body 104. It is also envisioned, however, that the window 126 may span less than the entire transverse cross-sectional dimension D of the tubular body 104.

The window 126 extends into the tubular body 104 so as to define a proximal end wall 130, an (opposing) distal end wall 132, and an inner wall 134 that extends between the end walls 130, 132. More specifically, in the particular embodiment of the osteotomy device 100 seen in FIG. 1, the window 126 extends through the tubular body 104 in (generally) orthogonal relation to the longitudinal axis Y. Embodiments in which the window 126 may extend through the tubular body 104 in non-orthogonal relation to the longitudinal axis Y, however, are also contemplated herein.

To allow for extension of the medical instrument(s) 200 into the window 126, the proximal end wall 130 and/or the inner wall 134 defines one or more openings 136 that are in communication with the lumen(s) 114, which allows the medical instrument(s) 200 to extend through the lumen(s) 114 and into the window 126 (via the opening(s) 136) to access the treatment area 128. In the particular embodiment illustrated, the distal end wall 132 is closed (e.g., devoid of any openings 136), which provides an operative (bracing) surface that supports the target tissue T and limits continued distal advancement of the medical instrument(s) 200. Embodiments in which the distal end wall 132 may define one or more openings 136, however, are also contemplated herein.

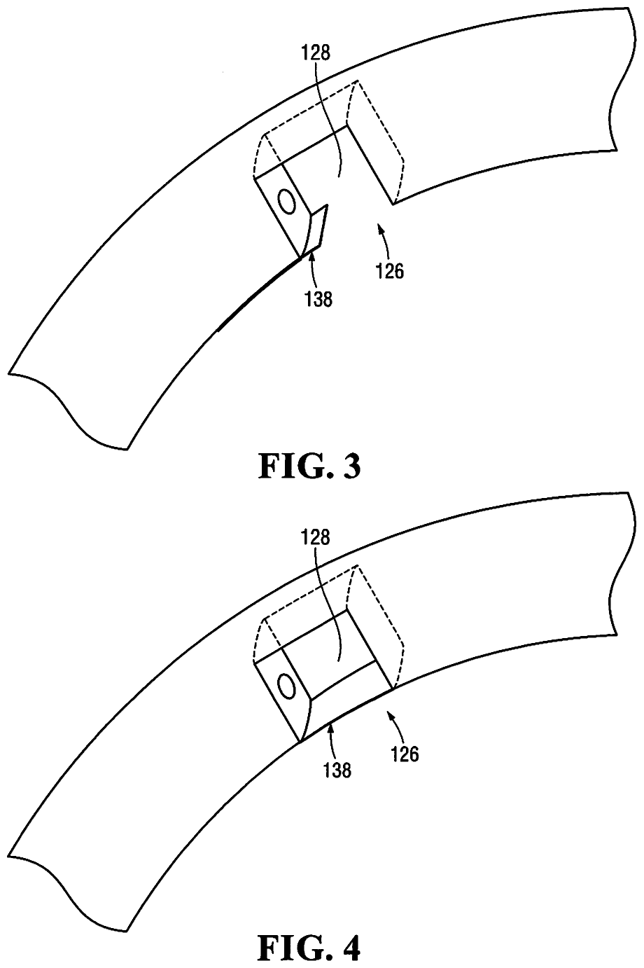

In certain embodiments of the disclosure, it is envisioned that the osteotomy device 100 may include a door 138 (or other such covering) that is configured to close the window 126 and thereby conceal the treatment area 128, as seen in FIGS. 3 and 4. In such embodiments, it is envisioned that the door 138 may be removably connected to the tubular body 104 and/or movable (e.g., pivotable, rotatable, slidable, etc.) in relation to the tubular body 104 such that the door 138 is repositionable between a first (open) position (FIG. 3), in which the window 126 is exposed, and a second (closed) position (FIG. 4), in which the door 138 closes (covers) the window 126 (FIG. 3 illustrates the door 138 as being partially open during movement from the first position to the second position).

In certain embodiments, such as that seen in FIG. 1, for example, the osteotomy device 100 further includes a handle 140 that is attached to the proximal end 106 of the tubular body 104 such that the handle 140 is located externally of the patient during the course of the surgical procedure to allow for access to and control over the handle 140 by the user. Although (schematically) illustrated as including a square or rectangular configuration, it is envisioned that the handle 140 may include any configuration suitable for the intended purpose of facilitating control over the osteotomy device 100 in the manner described herein. Additionally, while the handle 140 is shown as extending from the tubular body 104 in (generally) perpendicular (orthogonal) relation, it should be appreciated that the particular orientation of the handle 140 in relation to the tubular body 104 may be varied without departing from the present disclosure (e.g., based upon user preference, spatial requirements, the nature of the surgical procedure being performed, etc.).

It is envisioned that the handle 140 may be fixedly (e.g., non-removably) connected to the tubular body 104 or, alternatively, that the handle 140 may be releasably (removably, detachably) connected to the tubular body 104. For example, it is envisioned that certain medical instruments 200 may include a handle similar (or identical) in structure and/or function to the handle 140. In such embodiments and methods of use, the handle 140 may be omitted or removed from the tubular body 104 to eliminate redundancy and simplify the surgical system 10.

The handle includes a series of ports 142 that are configured for connection to and/or the receipt of one or more medical instruments 200. For example, in the particular embodiment illustrated, the handle 140 includes a (first) port 142i, a (second) port 142ii, and a (third) port 142iii. Depending upon the specific intended use of the surgical system 10, however, it should be appreciated that the number of ports 142 may be increased or decreased to support a wide variety of functionality. It is also envisioned that some or all of the ports 142 may be utilized during a given procedure and that the ports 142 may each be configured to accommodate more than one medical instrument 200, thereby allowing for a reduction in the overall size of the handle 140. One or more of the ports could alternatively be positioned at the proximal opening of the tubular body 104. The handle and/or ports could alternatively be longitudinally aligned with the proximal opening 110 rather than laterally positioned as shown in FIG. 1.

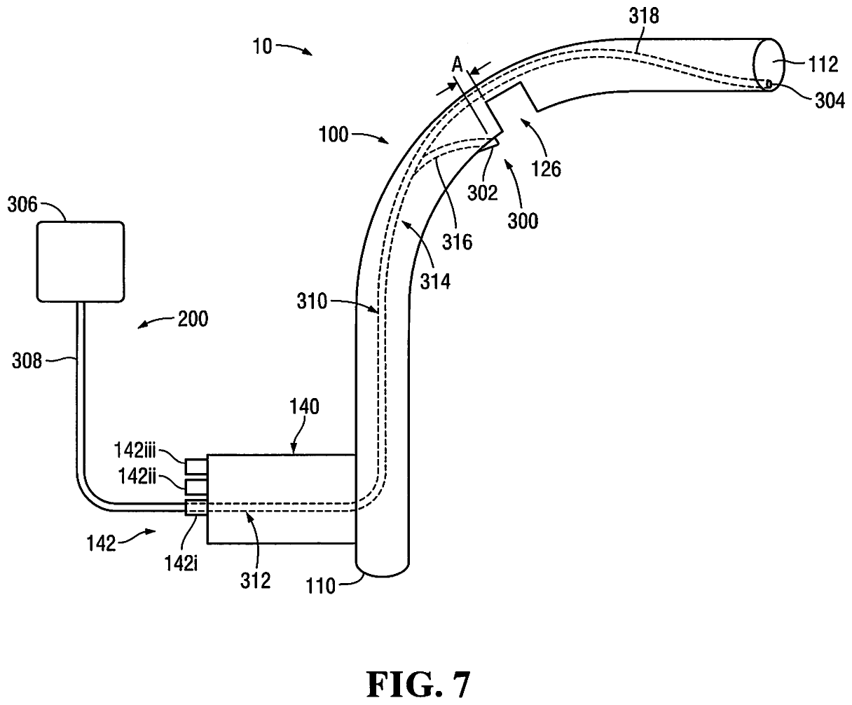

With continued reference to FIGS. 1-15, the surgical system 10 further includes an imaging system 300 (FIGS. 1, 7), which may be either fixedly, e.g., embedded in the wall of the device, or removably integrated into the osteotomy device 100 (as opposed to an external imaging system that is inserted into the patient separate and apart from osteotomy device 100). The imaging system 300 is configured to facilitate not only visualization of the target tissue T (FIG. 6A), the surgical site S, etc., but the capture of images, video, and other such data during the course of the surgical procedure. Note the target tissue for treatment utilizing the various systems discloses herein can include bone, soft tissue, calcified tissue, mucosa, etc.

More specifically, in the illustrated embodiment, the imaging system 300 includes a (first, proximal) imaging device 302 that is supported proximally of the window 126 and a (second, distal) imaging device 304 that is supported at (e.g., adjacent to, inside) the distal opening 112 to facilitate forward visualization during the surgical procedure (e.g., to guide the osteotomy device 100 during insertion and advancement towards the target tissue T). Although generally illustrated and described as endoscopic cameras throughout the present disclosure, it should be appreciated that the imaging devices 302, 304 may be configured in any manner suitable for the intended purpose of capturing images, video, or other such data. For example, in various embodiments, it is envisioned that the imaging devices 302, 304 may be configured to capture high-definition images and/or video, three-dimensional images and/or video, etc.

The imaging device 302 is spaced proximally a distance A (FIG. 7) from the window 126 that lies (substantially) within the range (approximately) 1 mm to (approximately) 50 mm (e.g., such that the imaging device 302 does not extend into or distally beyond the window 126). Distances A that lie outside the aforementioned range, however, are also envisioned herein (e.g., depending upon the visualization requirements of the particular procedure in which the surgical system 10 is employed). The proximal location of the imaging device 302 relative to the window 126 facilitates visualization of the target tissue T, which assists the clinician during reception of the target tissue T within the window 126 (and the treatment area 128) and allows the target tissue T and the surgical site S (as well as tissues located proximally and distally of the surgical site S) to be monitored during the surgical procedure. Thus, imaging device 302 forms a forward looking alligator eye proximal of the window 12.

The imaging devices 302, 304 are fixed (e.g., embedded within or otherwise secured to) the tubular body 104 in a manner that inhibits (if not entirely prevents) relative movement between the imaging devices 302, 304 and the tubular body 104. The imaging devices 302, 304, can be independent of the instruments inserted through the tubular body and out of contact with such instruments. Thus, in these embodiments, the instrument positioning and/or movement is independent of the position of the imaging devices 302, 304. Embodiments are also envisioned in which the imaging device 302 and/or the imaging device 304 may be movable in relation to the tubular body 104 (e.g., through the primary lumen 114 (FIG. 2) or one of the aforementioned ancillary lumens), as discussed in further detail below.

In the particular embodiment illustrated in FIG. 1, the imaging device 302 is secured to the outer surface 118 of the tubular body 104 such that the imaging device 302 extends laterally outward (protrudes) therefrom at an angle (up to and including 90°) and the imaging device 304 is flush-mounted to the distal end 108 of the tubular body 104. It should be appreciated, however, that the imaging devices 302, 304 may be oriented in any manner suitable for the intended purpose of facilitating the capture of images, video, and other such data in the manner described herein. For example, in alternate embodiments of the disclosure, the imaging device 302 may be flush-mounted to the tubular body 104 or may be recessed into the tubular body 104 (e.g., such that the outer surface 118 of the tubular body 104 is (generally) uniform) and/or the imaging device 304 can be recessed into the tubular body 104 or extend distally of the tubular body 104.

With reference to FIG. 8, in another embodiment, it is envisioned that the tubular body 104 may include a visualization port 144 formed in the outer wall 124 of the tubular body 104 that is located proximally of the window 126 to allow for the capture of images, video, or other such data through the visualization port 144. In such embodiments, it is envisioned that the imaging device 302 may be secured to the tubular body 104 at (e.g., adjacent to) the visualization port 144 or that the imaging device 302 may be movable through the tubular body 104 (e.g., through the primary lumen 114 (FIG. 2) or one of the aforementioned ancillary lumens) such that the imaging device 302 is positionable at (e.g., adjacent to) the visualization port 144.

In the embodiment seen in FIG. 8, the visualization port 144 is configured as an aperture 146 (or other such opening) that extends through the outer wall 124 of the tubular body 104 such that the imaging device 302 extends (or is extendable) into and/or through the visualization port 144 so as to protrude laterally from the tubular body 104. In alternate embodiments, however, it is envisioned that the visualization port 144 may be defined by one or more transparent (or otherwise non-opaque) sections 124t of material that are integrated into the outer wall 124 of the tubular body 104. In such embodiments, the imaging device 302 may be positioned internally of (e.g., behind) the transparent section(s) 124t (either fixedly or movably) so as to facilitate the capture of images, video, or other such data through the transparent section(s) 124t.

To support functionality of the imaging devices 302, 304, the imaging system 300 may include a variety of additional components. For example, in the particular embodiment seen in FIG. 7, the imaging system includes an image-collection controller 306, an external image-collection circuit 308, and internal image-collection circuit 310.

The external image-collection circuit 308 facilitates communication between the image-collection controller 306 and the internal image-collection circuit 310 and extends into the handle 140 (e.g., via the port 142i). In various embodiments of the disclosure, it is envisioned that the external image-collection circuit 308 may be either fixedly or releasably connected to the image-collection controller 306.

The internal image-collection circuit 310 extends through the tubular body 104 (e.g., via the primary lumen 114 (FIG. 2) or through one of the aforementioned ancillary lumens), thereby further integrating the imaging system 300 into the osteotomy device 100, and includes respective proximal and distal ends 312, 314. The proximal end 312 is connected to the external image-collection circuit 308 (e.g., within the handle 140) and the distal end 314 is connected to the imaging devices 302, 304 so as to permit visualization of the window 126 and the target tissue T (e.g., via the imaging device 304) and those tissues located distally of the target tissue T (e.g., via the imaging device 302). More specifically, in the particular embodiment illustrated, the distal end 314 of the internal image-collection circuit 310 includes a bifurcated configuration defining a (first) branch 316 that is connected to the imaging device 302 and a (second) branch 318 that is connected to the imaging device 304.

While the external image-collection circuit 308 and the internal external image-collection circuit 310 are illustrated as being separate, discrete structures in the particular illustrated embodiment seen in FIG. 7, it is also envisioned that the external image-collection circuit 308 and the internal external image-collection circuit 310 may be unitarily formed in certain embodiments such that the imaging system 300 includes a single image-collection circuit.

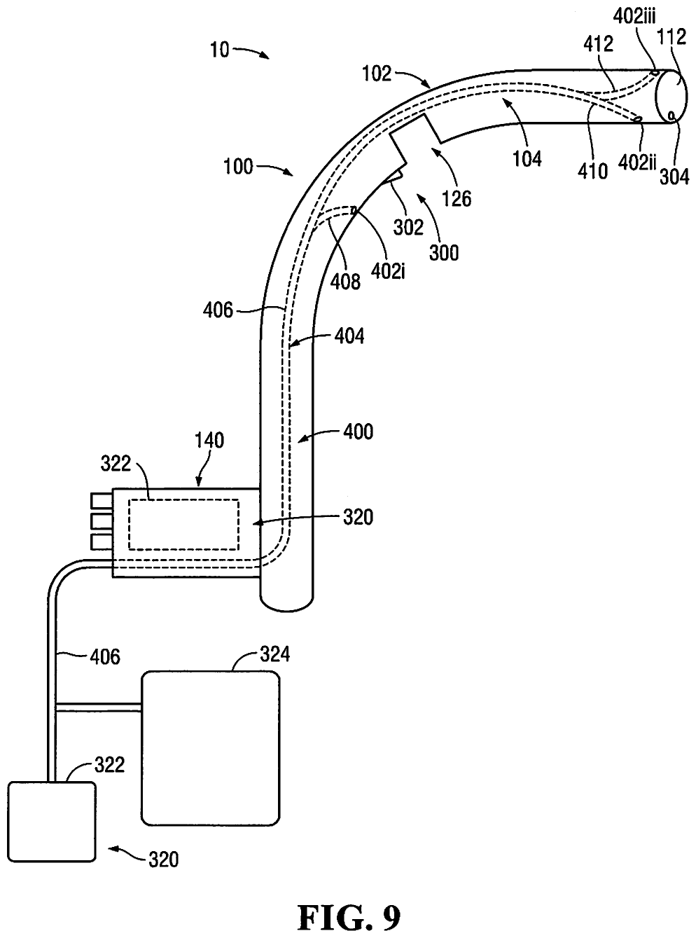

To support and/or enhance the capture of images, video, and other such data at the surgical site S (FIG. 6A), in certain embodiments of the disclosure, it is envisioned that the surgical system 10 may further include an illumination system 400 (FIG. 9) with one or more light sources 402 that are integrated into (embedded within, secured to, or movable through) the tubular body 104 of the osteotomy device 100. For example, in the particular embodiment illustrated, the imaging system 300 includes a first light source 402i that is fixedly secured to the tubular body 102 proximally of the window 126 and adjacent to the imaging device 302 and respective second and third light sources 402ii, 402iii are fixedly secured to the tubular body 102 distally of the window 126 and adjacent to the imaging device 304. More specifically, the light source 402ii is spaced proximally from the distal opening 112 and the light source 402iii is located at (or adjacent to) the distal opening 112. While the light sources 402ii, 402iii are shown as being (angularly) offset by (approximately) 180° in the illustrated embodiment (e.g., such that the light sources 402ii, 402iii are axially (longitudinally) misaligned so as to direct light in (generally) opposing directions), it is envisioned that the relative orientations of the light sources 402ii, 402iii, as well as the particular number of light sources 402, may be varied in alternate embodiments without departing from the scope of the present disclosure. For example, it is envisioned that the (angular) offset between the light sources 402ii, 402iii may be eliminated such that the light sources 402ii, 402iii are axially (longitudinally) aligned and/or that additional light sources 402 may be included at any location suitable for the intended purpose of supporting and/or enhancing the capture of images, video, and other such data.

It is envisioned that the (electrical) power may be communicated to the light sources 402 in any suitable manner. For example, in one embodiment, it is envisioned that the light sources 402 may be connected to the internal image-collection circuit 310 (FIG. 7). Additionally, or alternatively, it is envisioned that (electrical) power may be communicated to the light sources 402 via a separate conduit 404 (e.g., a cable 406 or the like), as seen in FIG. 9, that extends through the tubular body 104 (e.g., via the primary lumen 114 (FIG. 2) or through one of the aforementioned ancillary lumens) and the handle 140. In the particular embodiment illustrated, to facilitate the delivery of (electrical) power to each of the light sources 402, the conduit 404 includes a branched configuration defining a (first) branch 408 that is connected to the light source 402i, a (second) branch 410 that is connected to the light source 402ii, and a (third) branch 412 that is connected to the light source 402iii.

While the imaging devices 302, 304 and the light sources 402 are illustrated as being separate, discrete structures in FIG. 9, it is also envisioned that the imaging devices 302, 304 and the light sources 402 may be integrated in single units in alternate embodiments, and can be embedded in (built into) the wall of the device, without departing from the scope of the present disclosure.

It is envisioned that the imaging system 300 and the illumination system 400 may include (or may be connected to) any suitable power source 320 to thereby supply the necessary power to the imaging devices 302, 304 and the light sources 402. For example, it is envisioned that the imaging system 300 may include (or may be connected to) an external battery 322 via the conduit 404 or any suitable transmission media (e.g., one or the image-collection circuits 308, 310 (FIG. 7), one or more flexible printed circuits, a wiring harnesses, etc.). As seen in FIG. 9, the transmission media (e.g., the conduit 404) may extend through the tubular body 104 (e.g., via the primary lumen 114 (FIG. 2) or one of the aforementioned ancillary lumens) and may also be utilized to support the communication of images, video, or other such data from the imaging devices 302, 304 to a visual display 324, the image-collection controller 306 (FIG. 7), etc. Alternatively, it is envisioned that the power source 320 may be integrated into the osteotomy system 1 (e.g., the handle 140) to so as to further integrate the imaging system 300 (and/or the visualization system 400) into the osteotomy device 100.

With reference again to FIG. 1, in certain embodiments of the disclosure, the surgical system 10 further includes an aspiration system 500, which may be either fixedly or removably integrated into the osteotomy device 100. The aspiration system 500 includes an aspiration controller 502, an external aspiration tube 504, and an internal aspiration tube 506.

The external aspiration tube 504 is in fluid communication with the aspiration controller 502 and extends therefrom into the handle 140 (e.g., via the port 142ii). In various embodiments of the disclosure, it is envisioned that the external aspiration tube 504 may be either fixedly or releasably connected to the aspiration controller 502. The internal aspiration tube 506 is in fluid communication with external aspiration tube 504 (e.g., via the port 142ii) and extends into the handle 140 and through the tubular body 104 (e.g., via the primary lumen 114 or one of the aforementioned ancillary lumens), thereby integrating the aspiration system 500 into the osteotomy device 100. More specifically, the internal aspiration tube 506 is configured such that a distal end 508 thereof extends into the window 126 to facilitate the application of aspiration to the target tissue T (FIG. 6A) and/or the surgical site S and the removal of particulate, debris, etc., from the patient (e.g., via one or more openings formed in the distal end 508).

While the external aspiration tube 504 and the internal aspiration tube 506 are illustrated as being separate, discrete structures in the particular illustrated embodiment seen in FIG. 1, it is also envisioned that the external aspiration tube 504 and the internal aspiration tube 506 may be unitarily formed in certain embodiments such that a single tubular member extends from the aspiration controller 502 to the window 126 in the tubular body 104.

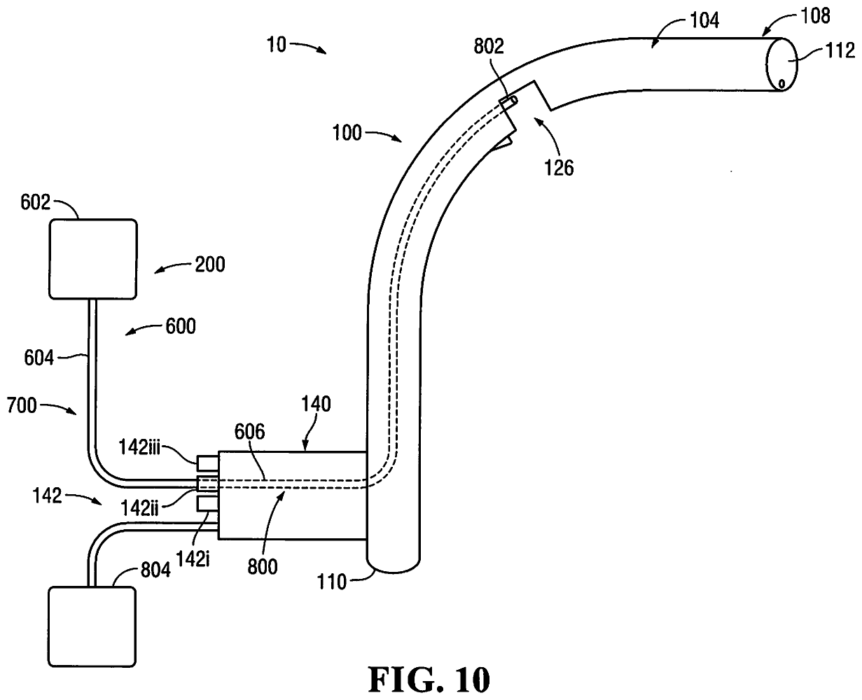

With reference to FIG. 10, additionally, or alternatively, it is envisioned that the surgical system 10 may further include a cauterization system 600 in which the medical instrument 200 includes a tissue treatment device 700 that is configured as a (monopolar or bipolar) cautery device 800. The cauterization system 600, with its cautery wire(s), may be either fixedly or removably integrated into the osteotomy device 100 and is configured to apply energy (e.g., heat) to the target tissue T (FIG. 6A). While the cautery device 800 is illustrated as including a generally cylindrical (e.g., tubular) configuration in FIG. 10, it is envisioned that the cautery device 800 may be configured in any manner suitable for the intended purpose of applying energy to the target tissue T in the manner described herein. For example, in an alternate configuration, it is envisioned that the cautery device 800 may include one or more individual cautery elements (e.g., wires), as described in further detail below. In some embodiments, one wire extends into the side window and the other wire extends out the distal end hole of the introducer.

To support operation of the cautery device 800, the cauterization system 600 includes a cauterization-communication controller 602 and one or more cauterization-communication circuits. In the particular embodiment illustrated, for example, the cauterization system 600 includes an external cauterization-communication circuit 604 and an internal cauterization-communication circuit 606 that is in (electrical) communication the external cauterization-communication circuit 604. The external cauterization-communication circuit 604 is in (electrical) communication with the cauterization-communication controller 602 and extends therefrom into the handle 140 (e.g., via the port 142i). The internal cauterization-communication circuit 606 is in (electrical) communication with the external cauterization-communication circuit 604 and extends into the handle 140 and through the tubular body 104 (e.g., via the primary lumen 114 or one of the aforementioned ancillary lumens), thereby integrating the cauterization system 600 into the osteotomy device 100. More specifically, in the particular embodiment illustrated in FIG. 10, the cautery device 800 is configured such that a distal end 802 thereof extends into the window 126 to cauterize the target tissue T (FIG. 6A). Embodiments are also envisioned, however, in which the cautery device 800 may extend to the distal end 108 of the tubular body 104 (e.g., such that the cautery device 800 is positioned within or adjacent to the distal opening 112) as are embodiments in which the osteotomy device 100 includes a (first) cautery device 800 is that extends into the window 126 and a (second) cautery device 800 that extends to the distal end 108 of the tubular body 104 such that cauterization may be applied simultaneously in a plurality of locations.

To facilitate the communication of energy to the distal end 802 of the cautery device 800, in certain embodiments, it is envisioned that the tubular body 104 may include non-insulated sections or components that terminate at various locations at, within, or adjacent to the window 126. It is also envisioned that the non-insulated sections or components may terminate at various locations at or adjacent to the distal opening 112.

While the external cauterization-communication circuit 604 and the internal cauterization-communication circuit 606 are illustrated as being separate, discrete structures in the particular illustrated embodiment seen in FIG. 10, it is also envisioned that the external cauterization-communication circuit 604 and the internal cauterization-communication circuit 606 may be unitarily formed in certain embodiments such that a single cauterization-communication circuit extends from the cauterization-communication controller 602 to the distal end 802 of the cautery device 800.

It is envisioned that the cautery device 800 may be inserted into the tubular body 104 through the handle 140 (e.g., via the port 142i) such that the cautery device 800 is electrically connectable to an external power source 804 (e.g., via electrical wiring extending through the handle 140). Alternatively, it is envisioned that the cautery device 800 may be connected to the power source 320 (FIG. 9) included in the handle 140 discussed above in connection with the imaging system 300.

In another embodiment of the disclosure, it is envisioned that the cautery device 800 may be configured for insertion into the tubular body 104 through the proximal opening 110 and connected to either the external power source 804 (e.g., via electrical wiring extending from the tubular body 104) or to the power source 320 (FIG. 9) included in the handle 140.

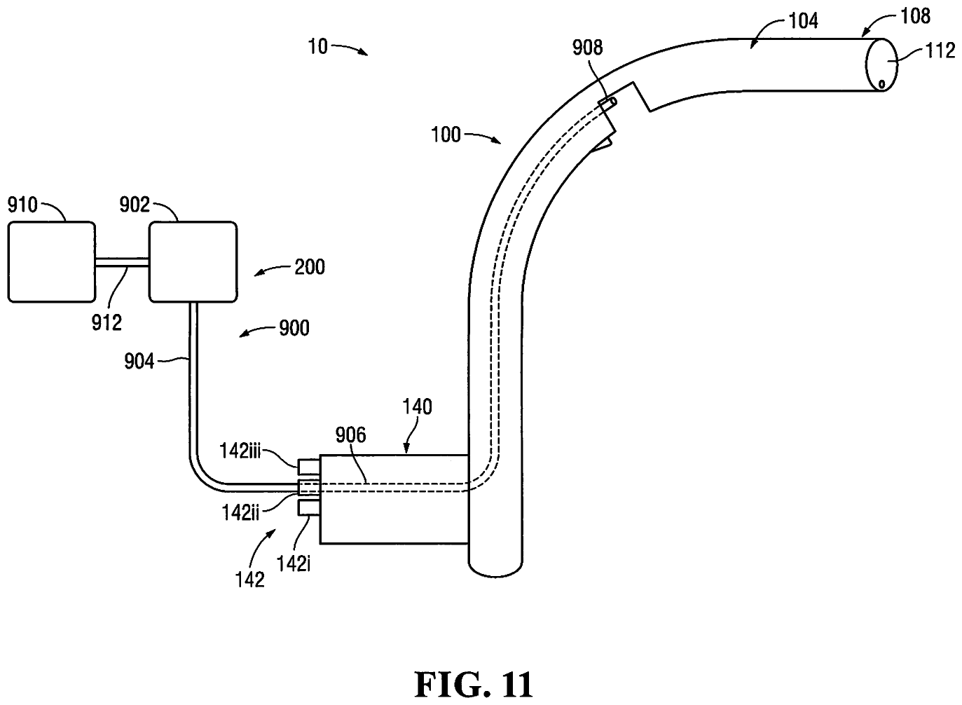

With reference to FIG. 11, additionally, or alternatively, it is envisioned that the surgical system 10 may further include an irrigation system 900, which may be either fixedly or removably integrated into the osteotomy device 100. The irrigation system 900 includes an irrigation controller 902, an external irrigation tube 904, and an internal irrigation tube 906. The irrigation tube can be built into the wall of the device 100

The external irrigation tube 904 is in fluid communication with the irrigation controller 902 and extends therefrom into the handle 140 (e.g., via the port 142iii). In various embodiments of the disclosure, it is envisioned that the external irrigation tube 904 may be either fixedly or releasably connected to the irrigation controller 902. The internal irrigation tube 906 is in fluid communication with the external irrigation tube 904 (e.g., via the port 142iii) and extends into the handle 140 and through the tubular body 104 (e.g., via the primary lumen 114 or one of the aforementioned ancillary lumens), thereby integrating the irrigation system 900 into the osteotomy device 100. More specifically, in the particular embodiment illustrated in FIG. 11, the internal irrigation tube 906 is configured such that a distal end 908 thereof extends into the window 126 to facilitate the communication (injection) of irrigation fluid to the target tissue T (FIG. 6A) and/or the surgical site S and the removal of particulate, debris, etc., from the patient (e.g., via one or more openings formed in the distal end 908). Embodiments are also envisioned, however, in which the internal irrigation tube 906 may extend to the distal end 108 of the tubular body 104 (e.g., such that the internal irrigation tube 906 is positioned within or adjacent to the distal opening 112) as are embodiments in which the osteotomy device 100 includes a (first) internal irrigation tube 906 that extends into the window 126 and a (second) internal irrigation tube 906 that extends to the distal end 108 of the tubular body 104 such that irrigation may be applied simultaneously in a plurality of locations.

It is envisioned that the irrigation system 900 may include (or may be connected to) a source of fluid 910. For example, in the illustrated embodiment, the irrigation system 900 is connected to the source of fluid 910 by a fluid conduit 912 that extends between the source of fluid 910 and the irrigation controller 902. Alternatively, it is envisioned that the fluid conduit 912 may extend between the source of fluid 910 and the port 142iii. It is also envisioned that the source of fluid 910 may be in fluid communication with one of the aforementioned ancillary lumens extending through the tubular body 104 or, alternatively, that an additional irrigation tube (not shown) may be provided within the tubular body 104.

With reference now to FIGS. 1 and 5, another embodiment of the tissue treatment device 700 will be discussed, which is configured as a tissue cutter 1000. The tissue cutter 1000 is configured to cut, reshape, excise, or otherwise alter the target tissue T (FIG. 6A) and is insertable through the tubular body 104 (e.g., via the proximal opening 110 and the primary lumen 114) in either a fixed or removable manner.

In the particular embodiment illustrated in FIG. 5, the tissue cutter 1000 includes a proximal end 1002 supporting a handle 1004 and an operative distal end 1006. The handle 1004 includes a (first, upper) handle element 1008 and a (second, lower) handle element 1010. The handle elements 1008, 1010 are connected via a joint 1012 such that the handle element 1010 is movable (e.g., pivotable) in relation to the handle element 1008 upon actuation of the handle 1004.

The distal end 1006 includes (integrated) imaging devices 1014, 1016 (e.g., endoscopic cameras) as well as an end effector 1018 and a series of openings 1020. More specifically, the distal end 1006 includes one or more (first) openings 1022 and one or more (second) openings 1024.

The imaging devices 1014, 1016 are similar (or identical) to the imaging devices 302, 304 (FIG. 1) discussed above in structure, mounting, and operation and facilitate visualization during use of the tissue cutter 1000. In the particular embodiment illustrated, the imaging devices 1014, 1016 are fixed (e.g., embedded within, e.g. built into the wall of the introducer or otherwise secured to) the tubular body 104 in a manner that inhibits (if not entirely prevents) relative movement between the imaging devices 1014, 1016 and the distal end 1006. Embodiments in which the imaging device 1014 and/or the imaging device 1016 may be movable in relation to the distal end 1006 are also contemplated herein.

To support imaging, the imaging devices 1014, 1016 may be connected to any suitable transmission media 1026. In the particular embodiment seen in FIG. 5, the transmission media 1026 is received by lumens 1028, 1030 that extend through the tissue cutter 1000, thereby further integrating visualization into the tissue cutter 1000.

During use, the tissue cutter 1000 is advanced through the tubular body 104 (FIG. 1) (e.g., via the proximal opening 110 and the primary lumen 114) until the imaging device 1014 is positioned distally of the window 126 in the tubular body 104 (e.g., adjacent to the distal opening 112 so as to facilitate forward visualization) and the imaging device 1016 is (generally) aligned with the window 126 so as to facilitate visualization of the target tissue T and/or the surgical site S. During such use, the tubular body 104 thus acts as a delivery member (sheath) for the tissue cutter 1000.

To facilitate operation of the end effector 1018, the tissue cutter 1000 includes a (first, upper) element 1032 that extends from (e.g., is connected to) the handle element 1008 and a (second, lower) element 1034 that extends from (e.g., is connected to) the handle element 1010 such that actuation of the handle 1004 causes relative axial (longitudinal) movement (e.g., sliding) of the elements 1032, 1034 along an interface 1036. More specifically, upon closure of the handle 1004 (e.g., approximation of the handle elements 1008, 1010), the element 1034 is retracted (relative to the element 1032), whereby respective distal ends 1038, 1040 of the elements 1032, 1034 are approximated to thereby act upon (e.g., cut) the target tissue T (FIG. 6A), which may be facilitated by the inclusion of one or more incisive edges or members on the distal end 1038 and/or the distal end 1040. Upon opening of the handle 1004 (e.g., separation of the handle elements 1008, 1010), the element 1034 is advanced (relative to the element 1032), whereby the respective distal ends 1038, 1040 of the elements 1032, 1034 are separated. The end effector 1018 is, thus, repositionable between opened and closed configurations via manipulation of the handle 1004.

In certain embodiments, such as that seen in FIG. 5, for example, it is envisioned that the handle 1004 may include a biasing member 1042 (e.g., a spring 1044 or the like) to bias the handle 1004 (and, thus, the end effector 1018) towards the open configuration.

In the particular embodiment illustrated, to support additional functionality, the tissue cutter 1000 includes the aforementioned ports 142 discussed in connection with the handle 140 (FIG. 1). Although illustrated as including three ports 142 (e.g., ports 142i, 142ii, 142iii), depending upon the specific intended use of the tissue cutter 1000 and the requirements of the surgical procedure being performed, it is envisioned that one or more of the ports 142i, 142ii, 142iii may be omitted and that one or more supplemental ports 142 may be added to support a wide variety of functionality. It is also envisioned that each of the ports 142i, 142ii, 142iii may be utilized during a given procedure, or that only certain of the ports 142i, 142ii, 142iii may be utilized.

The port 142i is configured to receive one or more conductive elements 1046 that extend from a power source 1048 to the end effector 1018 to thereby energize the end effector 1018 and facilitate cauterization of the target tissue T (FIG. 6A) (e.g., subsequent to cutting) if necessary or desired. For example, in the illustrated embodiment, the tissue cutter 1000 includes a pair of conductive elements 1046i, 1046ii that are respectively received by the lumens 1028, 1030 extend through the elements 1032, 1034, thereby integrating cauterization into the tissue cutter 1000 (for simplicity and clarity, only those portions of the conductive elements 1046, 1046ii extending externally of the tissue cutter 1000 are illustrated). While the power source 1048 is illustrated as being located externally of the tissue cutter 1000 in the illustrated embodiment, it is also envisioned that the power source 1048 may be integrated into the tissue cutter 1000 (e.g., into the handle 1004).

The port 142ii is configured for connection to an aspiration source 1050 to apply aspiration to the target tissue T (FIG. 6A) and/or the surgical site S through the opening(s) 1022 to thereby remove particulate, debris, etc., from the patient. It is envisioned that the port 142ii and the opening(s) 1022 may be connected by an aspiration lumen (e.g., a conduit, channel, etc.) that extends through the element 1034, thereby integrating aspiration into the tissue cutter 1000.

The port 142iii is configured for connection to an irrigation source 1052 to provide irrigation fluid to the target tissue T (FIG. 6A) and/or the surgical site S through the opening(s) 1024. It is envisioned that the port 142iii and the opening(s) 1024 may be connected by an irrigation lumen (e.g., a conduit, channel, etc.) that extends through the element 1034 (e.g. in (generally) parallel relation to the aspiration lumen, thereby integrating irrigation into the tissue cutter 1000.

In various embodiments of the disclosure, it is envisioned that certain functionality may be allocated between the osteotomy device 100 and the tissue cutter 1000. For example, it is envisioned that the cutting and cauterization may be performed by the tissue cutter 1000 while irrigation and aspiration may be performed by the osteotomy device 100. It is also envisioned that the osteotomy device 100 and the tissue cutter 1000 may include common (e.g., overlapping) functionality. For example, it is envisioned that visualization, irrigation, and/or aspiration may be performed by both the osteotomy device 100 and the tissue cutter 1000 during the course of the surgical procedure. To support robust functionality of the surgical system 10, it is thus envisioned that the ports 142 may be provided on the osteotomy device 100 and/or the tissue cutter 1000.

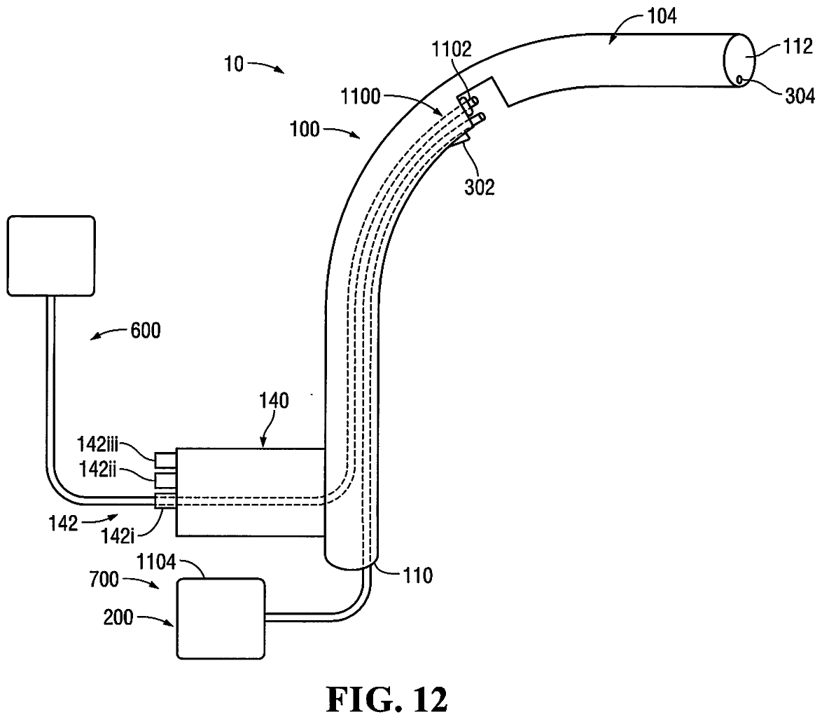

With reference now to FIGS. 6A, 6B, and 12, an alternate embodiment of the disclosure is illustrated in which the tissue treatment device 700 is configured as an ultrasonic or laser bone cutter 1100 that includes an operative distal end (tip) 1102. The bone cutter 1100 is connected to (in communication with) a controller 1104 that provides (ultrasonic or laser) energy to the operative distal end 1102 to facilitate (ultrasonic or laser) treatment of the target tissue T (FIG. 6A), which is shown as a portion P of a vessel wall V.

In the particular embodiment and method illustrated, the bone cutter 1100 is shown with the aforementioned cauterization system 600, each of which extends through the tubular body 104 (e.g., via the lumen 114 (FIG. 2) or one of the aforementioned ancillary lumens) and into the window 126 through corresponding openings 136i, 136ii (FIG. 6B) formed in the proximal end wall 130 thereof. For example, it is envisioned that the bone cutter 1100 may be inserted into the tubular body 104 via the proximal opening 110 and that the cauterization system 600 may be inserted into the tubular body 104 via the port 142i in the handle 140.

During treatment of the target tissue T, the collateral tissue C is shielded (protected) from the bone cutter 1100 and the cauterization system 600 by the tubular body 104 of the osteotomy device 100 via confinement of the applied treatment to the treatment area 128 defined by the window 126. During treatment, the target tissue T is monitored by the imaging device 302 located proximally of the window 126 and distal sections of the tissues are monitored by the imaging device 304. The tissue being treated, e.g., cut, is shielded with the window (slit/recessed region) which receives a tip of the cutting or other treatment instrument. The tip of the instrument is also shielded within the window 126. The treatment, e.g., drilling, is thus in a narrow area as the slit (window) protects surrounding tissue.

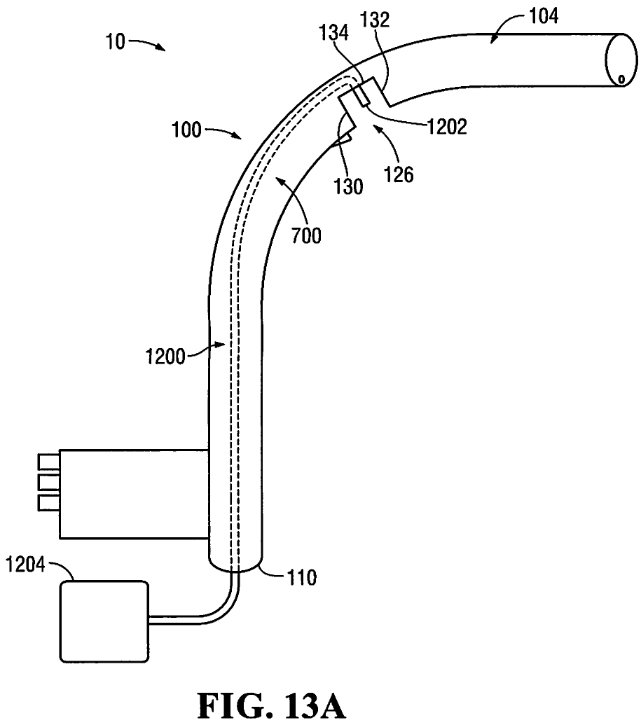

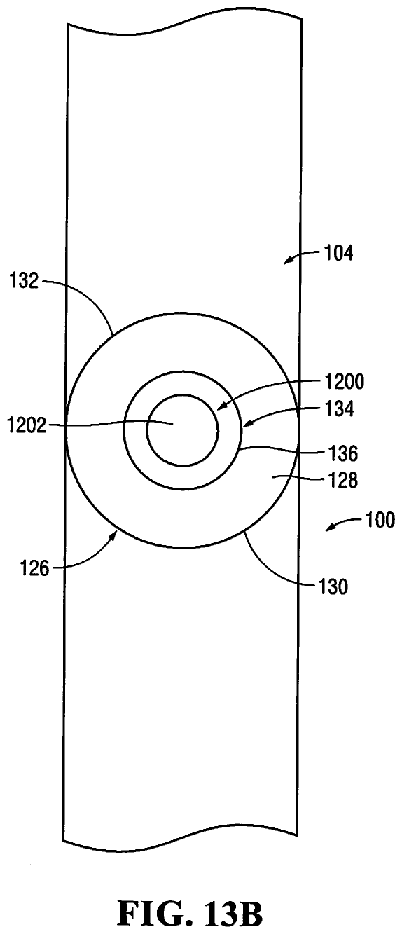

FIGS. 13A and 13B illustrate another embodiment of the disclosure in which the tissue treatment device 700 is configured as a bone drill 1200 including an operative distal end (tip) 1202. The bone drill 1200 is configured for insertion into the tubular body 104 (e.g., via the proximal opening 110) such that the operative distal end 1202 extends into the window 126, whereby the tubular body 104 of the osteotomy device 100 shields (protects) the collateral tissue C (FIG. 6A) during treatment of the target tissue T by confining the drilling operation to the treatment area 128 defined by the window 126.

In the particular embodiment illustrated, the distal end 1202 of the bone drill 1200 extends into the window 126 through an opening 136 that is positioned so as to (generally) bisect the inner wall 134. It should be appreciated, however, that the particular location of the opening 136 may be varied without departing from the scope of the present disclosure. For example, it is envisioned that the opening 136 may be located along the inner wall 134 such that the distance defined between the opening 136 and the proximal end wall 130 is less than or greater than the distance defined between the opening 136 and the distal end wall 132. In additional embodiments, it is also envisioned that the opening 136 through which the distal end 1202 of the bone drill 1200 extends may instead be formed in the proximal end wall 130 defined by the window 126.

In the particular embodiment illustrated, the bone drill 1200 is connected to (in communication with) a controller 1204 that is configured to regulate operation (e.g., power, speed, mode, etc.) of the bone drill 1200. Alternatively, however, it envisioned that the controller 1204 may be omitted and that the bone drill 1200 may be configured for manual operation.

With general reference now to FIGS. 6A, 14A, 14B, and 15, a method of using the surgical system 10 will be discussed in the context of a sinus procedure. In some embodiments, the method for use in a sinus procedure allows safe minimally invasive permanent expansion of a paranasal sinus (such as the maxillary sinus) by removing bone and mucosa around the ostium to enlarge it. The side slit (window) is placed such that it is in the ostium and over bone around the ostium to advance treatment devices. It should be appreciated that the surgical system 10 may be configured and utilized in a wide variety of surgical procedures in which it is desirable to treat (e.g., cut, reshape, excise, or otherwise alter) tissue.

Initially, the osteotomy device 100 is inserted into the patient's sinuses and is advanced towards the surgical site S (FIG. 6A), which can be monitored using the imaging system 300 (e.g., the imaging devices 302, 304). Upon reaching the target tissue T, under visualization provided by the imaging device 302 (and the light source(s) 402 (FIG. 9)), the target tissue T is inserted into the window 126 such that the target tissue T is positioned within the treatment area 128 (and the primary lumen 114).

Thereafter, one or more of the medical instruments 200 (e.g., the tissue treatment device 700) may be utilized to either directly treat the target tissue T or support treatment of the target tissue T. For example, in the particular embodiment illustrated, the bone cutter 1100 and the bone drill 1200 are inserted into the tubular body 104 via the proximal opening 110 and the aspiration system 500 and the irrigation system 900 are inserted into the tubular body 104 through the handle 140 via the ports 70 and 80, respectively. It should be appreciated, however, that dependent upon the particular surgical procedure being performed, one or more of the bone cutter 1100, the bone drill 1200, the aspiration system 500, and the irrigation system 900 may be omitted and/or replaced by one or more other medical instruments 200. For example, in certain surgical procedures, it is envisioned that the bone cutter 1100 and/or the bone drill 1200 may be replaced with or supplemented by the cauterization system 600 (FIG. 10), which may be inserted into the tubular body 104 through any suitable port 142 on the handle and through the primary lumen 114. It is thus envisioned that the ports 142 and the primary lumen 114 may be used to accommodate more than one medical instrument 200, thereby allowing for a reduction in the overall size of the osteotomy device 100, as discussed above.

Following insertion into the tubular body 104, the medical instruments 200 are advanced through the primary lumen 114 towards the window 126 and the target tissue T. More specifically, in the illustrated embodiment, the bone cutter 1100, the bone drill 1200, and the irrigation system 900 are advanced through the tubular body 104 and into the treatment area 128 through corresponding openings 136i, 136ii, 136iii formed in the proximal end wall 130 of the window 126 and the aspiration system 500 is advanced through the tubular body 104 and into the treatment area 128 through an opening 136iv formed in the inner wall 134 of the window 126, thus integrating the bone cutter 1100, the bone drill 1200, the irrigation system 900, and the aspiration system 500 into the osteotomy device 100.

As indicated above, it is envisioned that one or more of the medical instruments 200 (e.g., the aspiration system 500 and/or the irrigation system 900) may be fixedly (e.g., non-removably) integrated into the osteotomy device 100, thereby obviating any need for insertion and advancement of the medical instruments 200 in such embodiments during the surgical procedure.

During treatment (alteration) of the target tissue T (FIG. 6A), the bone cutter 1100 and the bone drill 1200 are operated via signals communicated from the controllers 1104, 1204, respectively. Additionally, on an as-needed basis, fluid is communicated through the irrigation system 900 (e.g., via the irrigation controller 902 and the irrigation tubes 904, 906) and aspiration is applied by the aspiration system 500 (e.g., via the aspiration controller 502 and the aspiration tubes 504, 506) to remove particulate, debris, etc., from the surgical site S (e.g., contemporaneously (simultaneously) with alteration of the target tissue T). Additionally, treatment of the target tissue T is monitored using the imaging system 300 (e.g., the imaging device 302), which may be supplemented by light provided via the light source(s) 402 (FIG. 9). The images, video, and other such data collected by the imaging system 300 may be displayed and/or processed by communicating the data through the image collection circuits 308, 310 to the image-collection controller 306.

Following treatment of the target tissue T, each of the medical instruments 200 (e.g., the bone drill 1200, the bone cutter 1100, the irrigation system 900, and the aspiration system 500) can be deactivated. In those embodiments of the disclosure in which the medical instrument(s) 200 are removably integrated into the osteotomy device 100, the medical instrument(s) 200 can be then be withdrawn and removed from the tubular body 104 and the osteotomy device 100 can be withdrawn and removed from the patient.

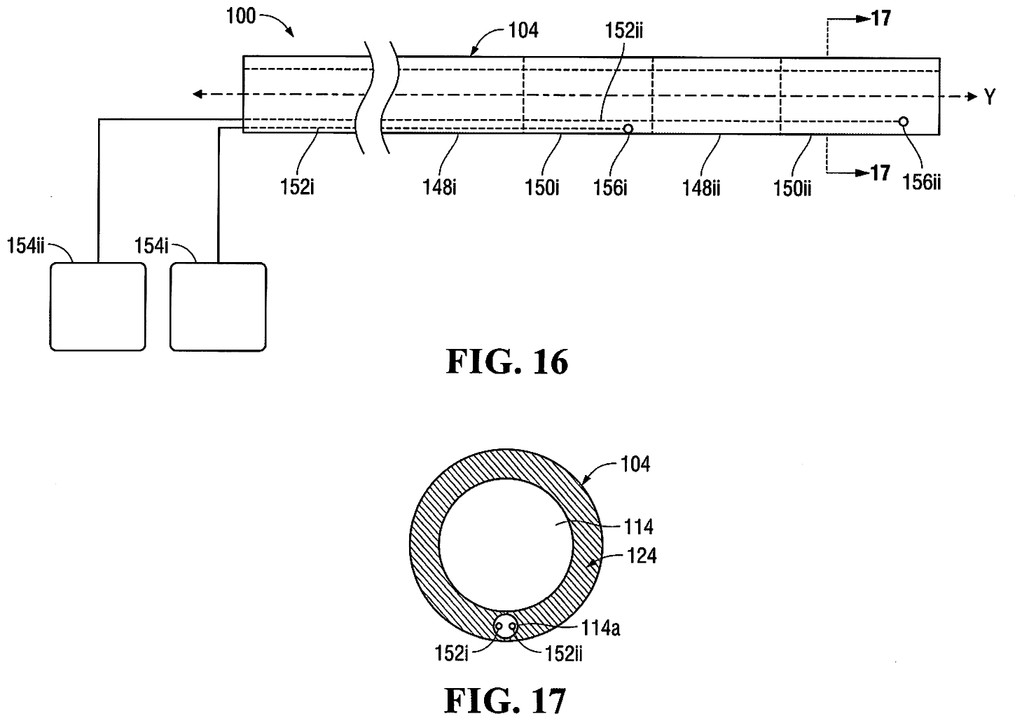

To facilitate access to various locations within the patient's sinuses or other surgical site S (e.g., a blood vessel, etc.) and/or anchoring (bracing) of the osteotomy device 100 within the patient, it is envisioned that the osteotomy device 100 and the medical instrument(s) 200 (e.g., the tissue treatment device 700) may be configured for deflection (reconfiguration). For example, it is envisioned that the osteotomy device 100 and the medical instrument(s) 200 (e.g., the tissue treatment device 700) may include (e.g., may be formed partially or entirely from) one or more flexible (e.g., malleable, bendable, and/or resilient) materials. For example, the treatment devices, e.g., the drills can be malleable to go around the curve of the introducer. Additionally, or alternatively, it is envisioned that the osteotomy device 100 and the medical instrument(s) 200 may be configured for controlled articulation to deflect one or more segments (sections) thereof. For example, FIGS. 16-18 illustrate an embodiment of the osteotomy device 100 in which the tubular body 104 includes a plurality of inactive (passive) segments 148 and a plurality of active (steerable, deflectable, articulable) segments 150. The active segments 150 are connected to a plurality of pull wires 152 and are spaced along the longitudinal axis Y of the tubular body 104. The pull wires can be embedded within or substantially within the wall of the tubular body 104. A mechanism operatively connected to the pull wires can be actuated to shorten or lengthen one or more of the pull wires to actively steer the device 100.

In the particular embodiment illustrated, the osteotomy device 100 includes a first inactive segment 148i; a first active segment 150i that is located distally of the segment 148i; a second inactive segment 148ii that is located distally of the segment 150i; and a second active segment 150ii that is located distally of the segment 148ii. Thus, in the particular embodiment illustrated, the inactive segments 148 and the active segments 150 are arranged in a staggered pattern in which the tubular body 104 alternates between inactive segments 148 and active segments 150. It should be appreciated, however, that the particular arrangement of the inactive segments 148 and active segments 150 may be varied without departing from the scope of the present disclosure. For example, embodiments are also envisioned in which the tubular body 104 may include two or more inactive segments 148 or active segments 150 that are arranged in successive (e.g., adjacent) relation.

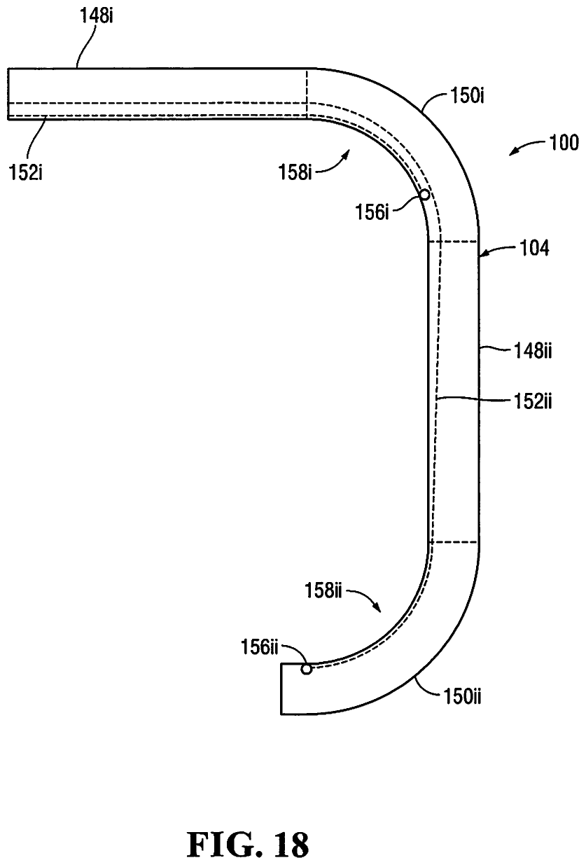

In the particular embodiment shown, each active segment 150 is connected to a corresponding (single) pull wire 152 that extends through (e.g., within) the outer wall 124 of the tubular body 104 such that the number of pull wires 152 corresponds to the number of active segments 150. More specifically, the osteotomy device 100 includes a first pull wire 152i that is connected to the first active segment 150i and a second pull wire 152ii that is connected to the second active segment 150ii. Upon the application of an axial (pulling) force to each of the pull wires 152, the corresponding active segment 150 is deflected (articulated) to thereby reconfigure (actively steer) the osteotomy device 100 between a first (initial, normal) configuration (FIG. 16), in which the tubular body 104 includes a (generally) linear configuration, and a second (subsequent, deflected) configuration (FIG. 18), in which the tubular body 104 includes a non-linear configuration. The use of a single pull wire 152 in connection with each active segment 150 reduces the requisite number of pull wires 152, thus reducing complexity in both construction and operation of the osteotomy device 100.

In the particular embodiment illustrated, the tubular body 104 includes one or more ancillary lumens 114a that are configured to receive the pull wires 152. The ancillary lumen(s) 114a are discrete from the primary lumen 114 and extend within the outer wall 124 such that the pull wires 152 are embedded within and are integrated into the tubular body 104. Although shown as including a single ancillary lumen 114a in the embodiment illustrated in FIGS. 16-18, embodiments are also envisioned in which the number of ancillary lumens 114a corresponds to that of the pull wires 152 such that each pull wire 152 is located (embedded) within a dedicated ancillary lumen 114a.

While the ancillary lumen 114a is illustrated as including a (generally) linear configuration that extends in (generally) parallel relation to the primary lumen 114 in the particular embodiment seen in FIGS. 16-18, it is also envisioned that the tubular body 104 may include one or more ancillary lumens 114a that are non-linear (e.g., tortuous, segmented) in configuration in various embodiments of the disclosure, as described in further detail below.

To facilitate the application of axial force to the pull wires 152, in certain embodiments, the osteotomy device 100 may include (or may be connected to) a plurality of corresponding activating mechanisms 154 (e.g., such that the number of pull wires 152 corresponds to the number of activating mechanisms 154). In the particular embodiment illustrated, the osteotomy device 100 includes a (first) activating mechanism 154i that is connected to the pull wire 152i and a (second) activating mechanism 154ii that is connected to the pull wire 152ii. The activating mechanisms 154 may include any structure or mechanism suitable for the intended purpose of applying the axial force to the pull wires 152 required to deflect the tubular body 104 as necessary or desired, such as, for example, rotating wheels, pulley systems, or the like. In certain embodiments, it is envisioned that the active segments 150, the pull wires 152, and the activating mechanisms 154 may be configured (and connected) such that each pull wire 152 may be individually acted upon to deflect (steer) the corresponding segment 150 in a single direction only.

The pull wires 152i, 152ii are connected to the segments 150i, 150ii at connection points 156i, 156ii (in addition to the activating mechanism 154i, 154ii), respectively, so as to facilitate reconfiguration of the osteotomy device 100 between the first configuration (FIG. 16) and the second configuration (FIG. 18). More specifically, upon reconfiguration of the osteotomy device 100, the active segments 150i, 150ii define respective first and second bends 158i, 158ii, which may be either substantially similar (e.g., identical) or dissimilar depending, for example, upon the particular configuration of the segments 150i, 150ii, the materials of construction used in the osteotomy device 100, the particular requirements of the osteotomy device 100 dictated by the surgical procedure, etc. Although the bends 158i, 158ii are each illustrated as being (approximately) equal to 90 degrees in FIG. 18, depending upon the particular configuration of the segments 150i, 150ii, the requirements of the surgical procedure, the particular anatomy of the patient, the structure and location of the surgical site S (FIG. 6A), etc., it is envisioned that the bends 158i, 158ii may lie substantially within the range of approximately 0 degrees to approximately 360 degrees.

In the particular embodiment illustrated, the connection points 156i, 156ii are shown as being in (general) angular alignment (e.g., along a circumference of the tubular body 104), which facilitates deflection of the segments 150i, 150ii in similar (e.g., identical) directions, as seen in FIG. 18. It is also envisioned, however, that the connection points 156i, 156ii may be angularly offset so as to facilitate deflection of the segments 150i, 150ii in dissimilar directions. For example, the connection points 156i, 156ii may be oriented in (generally) diametric opposition such that the bends 158i, 158ii respectively defined by the segments 150i, 150ii curve in (generally) opposite directions.

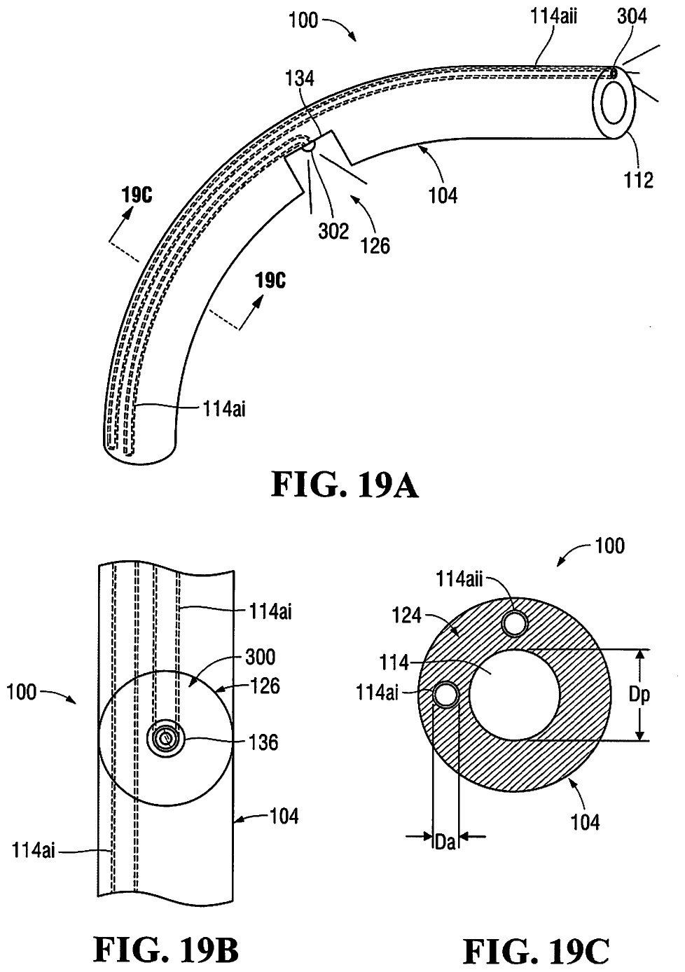

With reference now to FIGS. 19A-23C, a variety of alternate embodiments of the osteotomy device 100 will be discussed. Each of the embodiments discussed herein below is substantially similar to the aforedescribed osteotomy device 100 and, accordingly, will only be discussed with respect to any differences therefrom in the interest of brevity.

As mentioned above, it is envisioned that the tubular body 104 may include one or more ancillary lumens 114a that extend in (generally) parallel, discrete relation to the primary lumen 114. Throughout the present disclosure, the primary lumen 114 is illustrated as defining a (first) transverse cross-sectional dimension (e.g., a diameter) Dp and the ancillary lumens 114a are illustrated as defining a (second) transverse cross-sectional dimension (e.g., a diameter) Da that is less than the transverse cross-sectional dimension (e.g., a diameter) Dp, wherein the transverse cross-sectional dimension Da of each ancillary lumen 114a is (approximately) equal. It should be appreciated, however, that the particular transverse cross-sectional dimensions defined by the primary lumen 114 and the ancillary lumens 114a may be altered in various embodiments without departing from the scope of the present disclosure. For example, embodiments are envisioned in which the transverse cross-sectional dimensions Dp, Da may be (approximately) equivalent, as are embodiments in which the transverse cross-sectional dimension Da may exceed the transverse cross-sectional dimension Dp as well as embodiments in which the transverse cross-sectional dimensions Da defined by the ancillary lumens 114a may be unequal.

In the particular embodiment seen in FIGS. 19A-19C, for example, the tubular body 104 includes (first and second) ancillary lumens 114ai, 114aii that extend within the outer wall 124 thereof and are configured to receive (or otherwise accommodate) the imaging system 300 (e.g., the imaging devices 302, 304), which facilitates use of the primary lumen 114 to accommodate the tissue cutter 1000 (FIG. 5), the bone cutter 1100 (FIG. 12), the bone drill 1200 (FIG. 13A), etc. More specifically, the ancillary lumen 114ai is configured to receive the imaging device 302 (as well as any supporting circuitry, wiring, connectors, etc.) such that the imaging device 302 extends into the window 126 through the opening 136 formed in the inner wall 134, which is positioned so as to (generally) bisect the inner wall 134, and the ancillary lumen 114aii is configured to receive the imaging device 304 (as well as any supporting circuitry, wiring, connectors, etc.) such that the image device 304 is supported at (e.g., adjacent to, inside) the distal opening 112.

In the particular embodiment illustrated in FIGS. 19A-19C, the imaging devices 302, 304 are embedded (fixed) within the ancillary lumens 114ai, 114aii, respectively. It is also envisioned, however, that the imaging devices 302, 304 may be movable through the ancillary lumens 114ai, 114aii, respectively (e.g., to allow for positioning of the imaging device 302 at (e.g., adjacent to) the window 126).

In the particular embodiment illustrated, the ancillary lumens 114ai, 114aii are spaced by an angular distance of (approximately) 90°. It should be appreciated, however, that the particular relative orientation of the ancillary lumens 114ai, 114aii may be varied in alternate embodiments without departing from the scope of the present disclosure. For example, it is envisioned that the ancillary lumens 114ai, 114aii may be spaced by an angular distance of (approximately) 180° such that the ancillary lumens 114ai, 114aii are positioned in (generally) diametric opposition on opposite sides of the primary lumen 114.

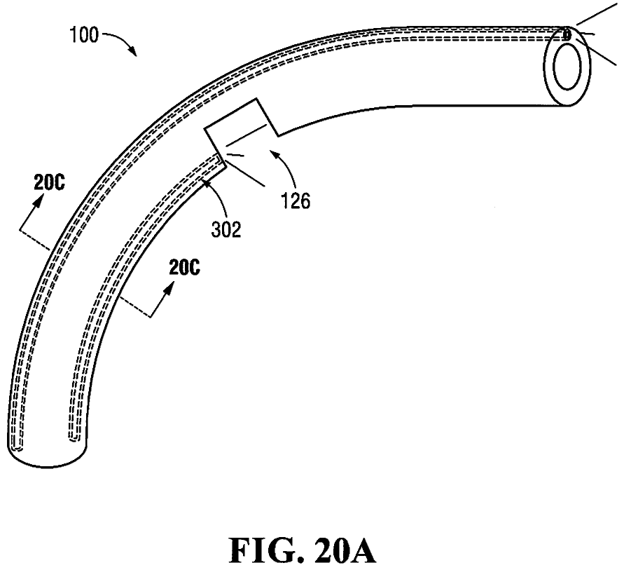

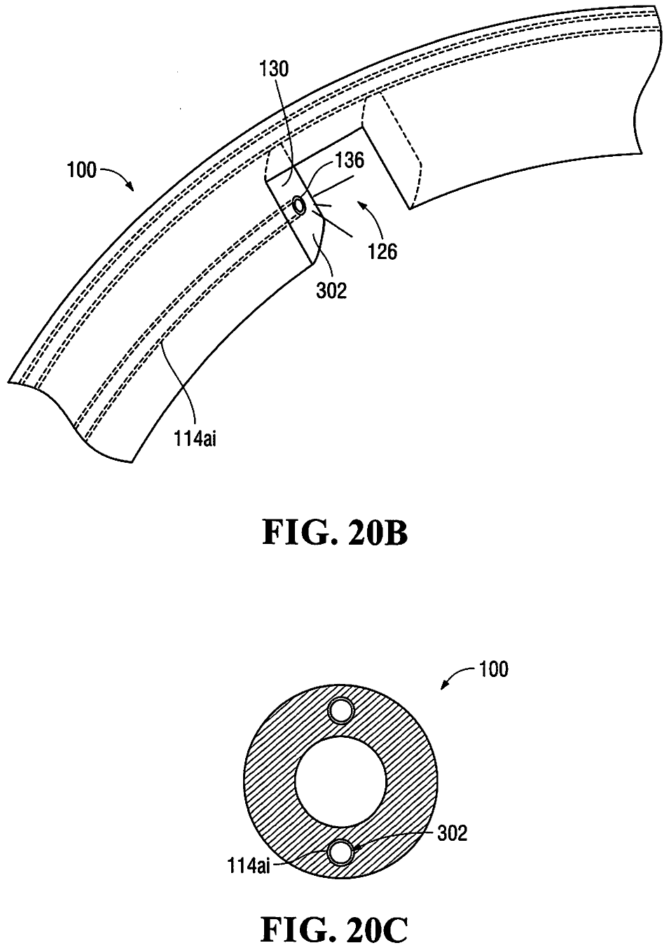

FIGS. 20A-20C illustrate another embodiment of the osteotomy device 100 in which the imaging device 302 extends into the window 126 through the ancillary lumen 114ai and the opening 136, which is formed in the proximal end wall 130 defined by the window 126.

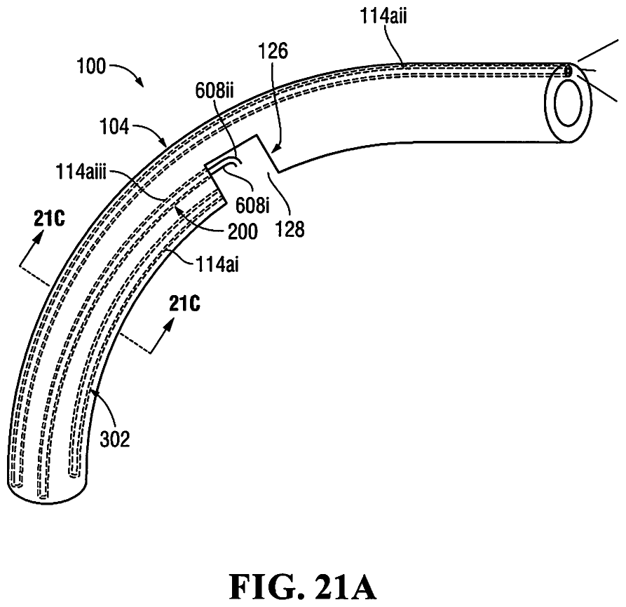

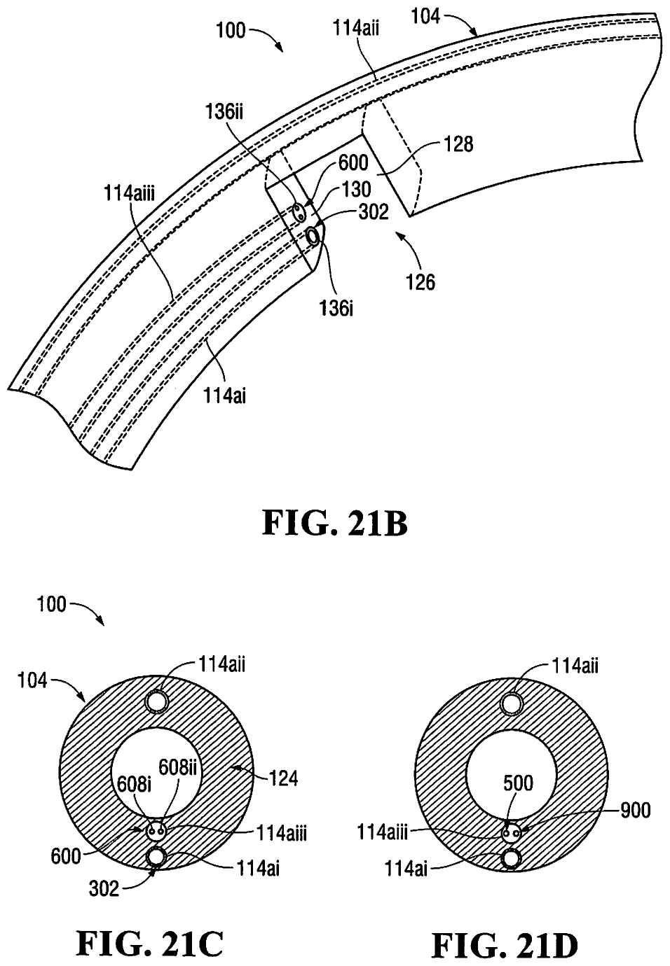

FIGS. 21A-21C illustrate another embodiment of the osteotomy device 100 in which the tubular body 104 further includes a (third) ancillary lumen 114aiii that is configured to receive one of the aforementioned medical instruments 200. Although illustrated as receiving the cauterization system 600, it should be appreciated that the ancillary lumen 114aiii may be utilized to accommodate the aspiration system 500 (FIG. 1) and/or the irrigation system 900 (FIG. 11), etc., in additional embodiments of the disclosure. In the illustrated embodiment, rather than the cautery device 800 discussed above in connection with FIG. 10, the cauterization system 600 includes (first and second) cautery elements (e.g., wires) 608i, 608ii.

The ancillary lumens 114ai, 114aiii extend through the outer wall 124 of the tubular body 104 and respectively terminate in the respective openings 136i, 136ii formed in the proximal end wall 130 defined by the window 126 such that the imaging device 302 extends into the window 126 (and the treatment area 128) through the opening 136i and the cautery wires 608i, 608ii extend into the window 126 (and the treatment area 128) through the opening 136ii. In an alternate embodiment, it is envisioned that the cautery wire 608i may extend into the window 126 through the opening 1136ii and that the cautery wire 608ii may continue distally to the distal opening 112. The cautery wires can be built into the wall of the introducer in some embodiments.

In the particular embodiment illustrated, the ancillary lumens 114ai, 114aiii are spaced from the ancillary lumen 114aii by an angular distance of (approximately) 180° such that the ancillary lumens 114ai, 114aiii and the ancillary lumen 114aii are positioned in (generally) diametric opposition on opposite sides of the primary lumen 114. It should be appreciated, however, that the particular relative orientation of the ancillary lumens 114ai, 114aii, 114aiii may be varied in alternate embodiments without departing from the scope of the present disclosure. For example, it is envisioned that one or more of the ancillary lumens 114ai, 114aiii may be spaced from the ancillary lumen 114aii by an angular distance of less than 180° (e.g., (approximately) 90°).

In a variation on the embodiment seen in FIGS. 21A-21C, it is envisioned that the ancillary lumen 114aiii may be configured to receive more than one medical instrument 200. For example, as seen in FIG. 21D, rather than the cauterization system 600, it is envisioned that the ancillary lumen 114aiii may instead be configured to receive the aspiration system 500 and the irrigation system 900.

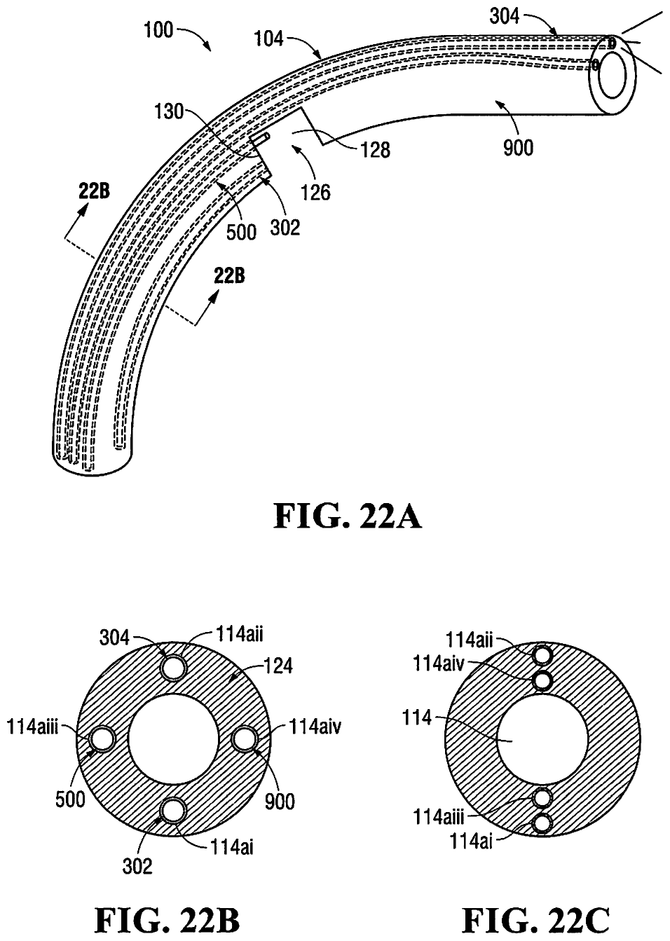

FIGS. 22A-22C illustrate another embodiment of the osteotomy device 100 in which the tubular body 104 further includes a (fourth) ancillary lumen 114aiv that is configured to receive one of the aforementioned medical instruments 200. More specifically, the osteotomy device 100 is configured such that the imaging device 302 and the aspiration system 500 extend into the window 126 to access the treatment area 128 through the ancillary lumens 114ai, 114aiii, respectively, and such that the imaging device 304 and the irrigation system 500 extend to the distal opening 112 through the ancillary lumens 114aii, 114aiv, respectively. It should be appreciated, however, that the ancillary lumens 114aiii, 114aiv may be configured and utilized to accommodate any of the medical instruments 200 described herein in alternate embodiments of the disclosure, either individually or in combination.

While the ancillary lumens 114aiii, 114aiv are illustrated as extending into the window 126 through the proximal end wall 130, it should be appreciated that either or both of the lumens 114aiii, 114aiv may instead extend into the window 126 through the inner wall 134 in alternate embodiments without departing from the scope of the present disclosure.

In the particular embodiment illustrated, the ancillary lumens 114ai-114aiv are spaced from each other by an angular distance of (approximately) 90°. It should be appreciated, however, that the particular relative orientation of the ancillary lumens 114ai-114aiv may be varied in alternate embodiments without departing from the scope of the present disclosure. For example, it is envisioned that the ancillary lumens 114aii, 114aiiv may be spaced from the ancillary lumens 114ai, 114aiii by an angular distance of (approximately) 180°, as seen in FIG. 22C, such that the ancillary lumens 114aii, 114aiv and the ancillary lumens 114ai, 114aiii are positioned in (generally) diametric opposition on opposite sides of the primary lumen 114.

In another embodiment of the disclosure, it envisioned that the irrigation system 900 may include a first internal irrigation tube 906i that extends into the window 126 (e.g., through the proximal end wall 130) and a second internal irrigation tube 906ii that continues distally to the distal opening 112.

It is also envisioned that aspiration may be applied via the proximal opening 110 (FIG. 1) (either in addition to or instead of aspiration applied via the window 126) such as, for example, in the event that aspiration to the window 126 is inhibited (or prevented) by a blockage, etc.

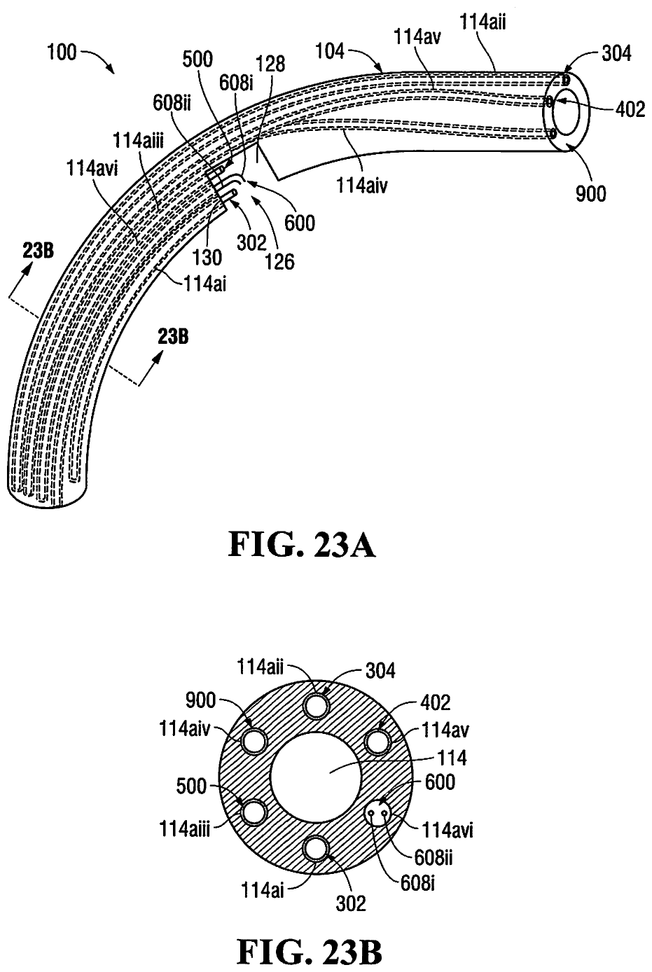

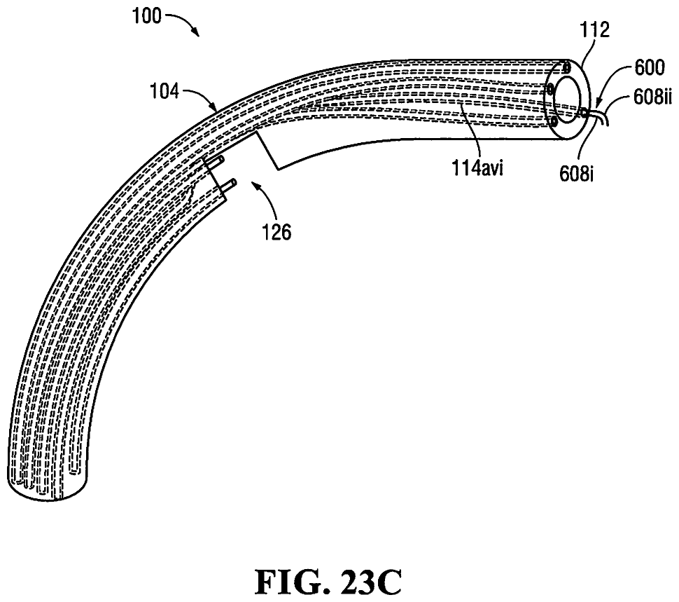

FIGS. 23A and 23B illustrate another embodiment of the osteotomy device 100 in which the tubular body 104 further includes a (fifth) ancillary lumen 114v and a (sixth) ancillary lumen 114vi, each of which is configured to receive one of the aforementioned medical instruments 200. More specially, the ancillary lumen 114ai is configured to receive the imaging device 302 (as well as any supporting circuitry, wiring, connectors, etc.), the ancillary lumen 114aii is configured to receive the imaging device 304 (as well as any supporting circuitry, wiring, connectors, etc.), the ancillary lumen 114aiii is configured to receive the aspiration system 500, the ancillary lumen 114aiv is configured to receive the irrigation system 900, the ancillary lumen 114av is configured to receive one or more of the aforementioned light sources 402, and the ancillary lumen 114avi is configured to receive the cauterization system 600, which, in the illustrated embodiment, includes the cautery elements 608i, 608ii. The ancillary lumens 114ai, 114aiii, and 114avi extend through the outer wall 124 of the tubular body 104 and the proximal end wall 130 such that the imaging device 302, the aspiration system 500, and the cauterization system 600 extend into the window 126 to access the treatment area 128.

In the particular embodiment illustrated, the ancillary lumens 114ai-114avi are spaced from each other by an (approximately) equal angular distance of (approximately) 60°. It should be appreciated, however, that the particular relative orientation of the ancillary lumens 114ai-114avi may be varied in alternate embodiments without departing from the scope of the present disclosure. For example, it is envisioned that angular spacing between the ancillary lumens 114ai-114avi may be unequal.

FIG. 23C illustrates a variation on the embodiment of the osteotomy device 100 illustrated in FIGS. 23A and 23B in which the cauterization the cauterization system 600 extends around the window 126 such that the cautery elements 608i, 608ii extend from the tubular body 104 through the distal opening 112. To facilitate passage of the cauterization system 600 through the distal opening 112 in the manner illustrated, it is envisioned that the ancillary lumen 114avi may include a non-linear (e.g., tortuous, segmented) configuration, as seen in FIG. 23C.

In some embodiments, the distal end hole could also allow irrigation and aspiration into the sinus, and biopsy, lesion removal, etc. without the need to remove or reposition the elongated device (introducer). This is facilitated under direct visualization via an imaging device positioned at or near the distal end hole, and one or more of aspiration, irrigation and/or cautery ports, etc. adjacent the distal end hole. Thus, such instruments/devices can be inserted through a distal end hole of the instrument, e.g., distal hole 112, for accessing tissue through the same introducer used to treat tissue at the side window wherein the introducer can remain in position if desired. Such tissue access and treatment at the distal end hole can be in addition or in lieu of the instruments for treatment at the side window. These instruments can each exit through the distal end hole or alternatively or additionally one or more ports can be provided adjacent the distal end of the elongated introducer for exit of one or more of the instruments. The port exit openings can be aligned with the distal end hole or alternatively recessed therefrom or extending distally therefrom. Separate lumens can be provided in some embodiments communicating with respective exit ports.

Where a range of values is provided, it is understood that each intervening value, to the tenth of the unit of the lower limit unless the context clearly dictates otherwise, between the upper and lower limit of that range and any other stated or intervening value in that stated range is encompassed within the scope of the present disclosure. The upper and lower limits of these smaller ranges which may independently be included in the smaller ranges is also encompassed by the scope of the present disclosure, subject to any specifically excluded limit in the stated range. Where the stated range includes one or both of the limits, ranges excluding either both of those included limits are also included within the scope of the present disclosure.

It will be understood that the above particular embodiments are shown and described by way of illustration only. The principles and the features of the present disclosure may be employed in various and numerous embodiments thereof without departing from the scope and spirit of the disclosure as claimed. The above-described embodiments illustrate the scope of the disclosure but do not restrict the scope of the disclosure and it should be understood by those skilled in the art that various changes may be made (and equivalents may be substituted) without departing from the true spirit and scope of the present disclosure. In addition, many modifications may be made to adopt a particular situation, material, composition of matter, process, process step or steps, to the objective spirit and scope of the present disclosure. All such modifications are intended to be within the scope of the claims appended hereto. For example, persons skilled in the art will understand that the elements and features shown or described in connection with one embodiment may be combined with those of another embodiment without departing from the scope of the present disclosure and will appreciate further features and advantages of the presently disclosed subject matter based on the description provided.

Throughout the present disclosure, terms such as “approximately,” “generally,” “substantially,” and the like should be understood to allow for variations in any numerical range or concept with which they are associated. For example, it is intended that the use of terms such as “approximately” and “generally” should be understood to encompass variations on the order of 25% (e.g., to allow for manufacturing tolerances and/or deviations in design).

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Additionally, as used herein, the singular forms “a,” “and,” and “the” should be understood to include plural references unless the context clearly dictates otherwise.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. A system for performing a surgical procedure on a patient, the system comprising:
an elongated introducer defining a lumen and including a window formed in an outer wall thereof in communication with the lumen;
a first imaging device fixedly supported on the elongated introducer distally of the window;
a second imaging device fixedly supported on the elongated introducer proximally of the window; and
at least one medical instrument configured for insertion into the patient through the elongated introducer such that the at least one medical instrument is positionable within the window, the window being configured to receive target tissue such that the target tissue extends into the lumen, whereby the elongated introducer shields collateral tissue from the at least one medical instrument to reduce any unintended effect on the collateral tissue during the surgical procedure.

2. The system of claim 1, wherein the first imaging device and the second imaging device are embedded in the outer wall of the elongated introducer.

3. The system of claim 1, wherein the at least one medical instrument includes a tissue treatment device configured for insertion through the lumen of the elongated introducer and into the window to facilitate treatment of the target tissue when the target tissue is positioned within the window.

4. The system of claim 3, wherein the tissue treatment device is configured as a tissue cutter or a bone drill.

5. The system of claim 3, wherein the at least one medical instrument further includes an aspiration system extending into the patient through the elongated introducer such that the aspiration system is integrated into the elongated introducer.

6. The system of claim 5, wherein the at least one medical instrument further includes an irrigation system extending into the patient through the elongated introducer such that the irrigation system is integrated into the elongated introducer.

7. The system of claim 6, wherein the at least one medical instrument further includes a cauterization system extending into the patient through the elongated introducer such that the cauterization system is integrated into the elongated introducer.

8. The system of claim 7, further including a handle connected to the elongated introducer.

9. The system of claim 1, further comprising a second medical instrument configured for insertion into the patient through the elongated introducer such that the second medical instrument is extendable distal of a distal end hole of the introducer and visualized by the first imaging device.

10. The system of claim 9, wherein the second medical instrument is one of an instrument for aspiration, irrigation or cautery.

11. The system of claim 9, further comprising one or more ports at a distal end of the elongated introducer for exit of the second medical instrument.

12. The system of claim 8, wherein the handle comprises:
a first port configured to receive the aspiration system such that the aspiration system extends into the elongate introducer through the first port;
a second port configured to receive the irrigation system such that the irrigation system extends into the elongate introducer through the second port; and
a third port configured to receive the cauterization system such that the cauterization system extends into the elongate introducer through the third port.

13. The system of claim 1, wherein the elongated introducer includes a proximal end and a distal end opposite to the proximal end, the elongated introducer defining a transverse cross-sectional dimension that is generally uniform between the proximal end and the distal end.

14. The system of claim 1, wherein the elongated introducer is steerable.

15. The system of claim 14, further comprising at least one wire substantially within the wall of the elongated introducer and movable by a mechanism to shorten or lengthen the at least one wire to steer the elongated introducer.

16. A system for use in performing a surgical procedure on a patient, the system comprising:
an elongated introducer including:
a window configured to receive target tissue such that the target tissue extends into the elongated introducer; and
a visualization port located proximally of the window; and
a first imaging device positioned adjacent to the visualization port such that the target tissue is viewable through the visualization port via the first imaging device;
a second imaging device positioned distally of the window; and
at least one medical instrument configured for insertion into the patient through the elongated introducer such that the at least one medical instrument is positionable within the window, the window being configured to receive the target tissue such that the target tissue extends into the elongated introducer, whereby the elongated introducer shields collateral tissue adjacent to the target tissue from the at least one medical instrument to reduce any unintended effect on the collateral tissue during the surgical procedure.

17. The system of claim 16, wherein the first imaging device extends through the visualization port such that the first imaging device protrudes laterally from the elongated introducer.

18. The system of claim 16, wherein the first imaging device and the second imaging device are embedded in an outer wall of the elongated introducer.

19. The system of claim 16, wherein the elongated introducer includes:
a primary lumen;
a first ancillary lumen discrete from the primary lumen;
a second ancillary lumen discrete from the primary lumen and the first ancillary lumen; and
a third ancillary lumen discrete from the primary lumen, the first ancillary lumen, and the second ancillary lumen.

20. The system of claim 19, further including:
a tissue treatment device configured for insertion into the primary lumen such that the tissue treatment device is positionable within the window to treat the target tissue;
an irrigation system extending through the first ancillary lumen such that the irrigation system is integrated into the elongated introducer;
an aspiration system extending through the second ancillary lumen such that the aspiration system is integrated into the elongated introducer; and
a cauterization system extending through the third ancillary lumen such that the cauterization system is integrated into the elongated introducer.

21. The system of claim 20, further including a handle connected to the elongated introducer, the handle comprising:
a first port configured to receive the irrigation system such that the irrigation system extends into the elongated introducer through the first port;
a second port configured to receive the aspiration system such that the aspiration system extends into the elongated introducer through the second port; and
a third port configured to receive the cauterization system such that the cauterization system extends into the elongated introducer through the third port.

22. The system of claim 20, wherein the elongated introducer is steerable.

23. The system of claim 20, further comprising at least one wire substantially within the wall of the elongated introducer and movable by a mechanism to shorten or lengthen the at least one wire to steer the elongated introducer.

24. A method of performing an osteotomy procedure on a patient's sinuses, the method comprising:
inserting an elongated introducer having a window configured to receive target tissue into the patient's sinuses;
visualizing tissue using an imaging device extending through the elongated introducer and positioned proximally of the window;
inserting a tissue treatment device into the patient's sinuses through the elongated introducer such that the tissue treatment device is positioned within the window;
positioning the elongated introducer such that the target tissue is received within the window and extends into the elongated introducer to thereby shield collateral tissue from the tissue treatment device to reduce any unintended effect on the collateral tissue during the osteotomy procedure; and
altering the target tissue using the tissue treatment device.

25. The method of claim 24, further comprising:
inserting an irrigation system into the patient's sinuses through the elongated introducer such that the irrigation system extends into the window; and
inserting an aspiration system into the patient's sinuses through the elongated introducer such that the aspiration system extends into the window.

26. The method of claim 24, further comprising simultaneously applying irrigation and aspiration during alteration of the target tissue via the tissue treatment device.

27. The method of claim 24, further comprising applying one or more of aspiration, irrigation and cautery adjacent a distal end hole of the elongated introducer, the distal end hole positioned distal of the window.

28. The method of claim 27, wherein the one or more of aspiration, irrigation and cautery is conducted under direct visualization via an imaging device adjacent the distal end hole.

29. The system of claim 24, wherein the elongated introducer is steerable.

30. The system of claim 29, further comprising at least one wire substantially within the wall of the elongated introducer and movable by a mechanism to shorten or lengthen'the at least one wire to steer the elongated introducer.
